(19) United States
(12) Patent Application Publication
(10) Pub. No.: US 2011/0008346 A1
(43) Pub. Date: Jan. 13, 2011

(54) BIOMARKERS FOR CARDIOVASCULAR DISEASE

(75) Inventor: Henricus Johannes Duckers, Rotterdam (NL)

Correspondence Address: REED SMITH LLP, 2500 ONE LIBERTY PLACE, 1650 MARKET STREET, PHILADELPHIA, PA, 19103, US

(73) Assignee: Erasmus University Medical Center Rotterdam, Rotterdam (NL)

(21) Appl. No.: 12/747,836

(22) PCT Filed: Dec. 12, 2008
(86) PCT No.: PCT/NL08/50799

§ 371 Date: Jul. 22, 2010

(30) Foreign Application Priority Data
Dec. 12, 2007 (NL) PCT/NL2007/050644

(51) Int. Cl. A61K 39/395; G01N 33/53; C12Q 1/68; C07K 14/00; C07H 21/02; C40B 30/04; C40B 40/00; C07K 16/18; A61K 31/7088; A61K 38/16; A61K 31/7105; A61K 31/711; A61P 9/10

(52) U.S. Cl. 424/136.1; 435/7.21; 435/6; 530/350; 536/23.1; 536/23.5; 435/7.1; 506/9; 506/13; 530/389.1; 514/44 R; 514/16.4; 424/172.1; 424/133.1; 514/44 A

(57) ABSTRACT

The present invention relates to a method of diagnosis or prognosis of cardiovascular disease in a subject, said method comprising the steps of detecting the presence of activated endothelial progenitor cells (EPCs) in a sample of a circulation fluid of said subject. The invention further relates to biomarkers for diagnosis or prognosis of cardiovascular disease in a patient, said biomarker comprising the expression product of a gene the expression of which is regulated during vasculogenesis.

### FIELD OF THE INVENTION

The present invention is in the field of diagnostics, more particular in the field of diagnosis, prognosis and treatment of cardiovascular disease. The present invention relates to biomarkers for diagnosis or prognosis of cardiovascular disease in a patient, to methods for the diagnosis or prognosis of cardiovascular disease in a subject, to a kit of parts for performing such methods, and to microarrays and diagnostic reagents useful in such methods. In particular the cardiovascular disease diagnosed relates to ischemic heart disease. The present invention further relates to methods of treating subjects (having an increased risk of) suffering from cardiovascular disease and to pharmaceutical compositions suitable for use in such treatment methods.

### BACKGROUND TO THE INVENTION

Ischemic heart disease is a disease characterized by reduced blood supply to the heart and is the major cause of morbidity and mortality in the Western world. Due to the intensive medical care required by patients, the disease constitutes a major investment of health care costs and health care infrastructure. Early diagnosis of the disease is difficult. In fact, there is no adequate test for the diagnosis of ongoing ischemia, nor for compensatory neovascularization.

Current diagnosis of ischemic heart disease is based on ergometric (exercise) testing or myocardial perfusion imaging. These techniques have limited sensitivity and specificity. A more reliable method would be to perform a coronary angiography. However, such percutaneous and invasive procedures are associated with considerable risks.

Therefore, there is a need for reliable biomarkers for the diagnosis and prognosis of ischemic heart disease.

### SUMMARY OF THE INVENTION

The present inventors have now discovered that gene expression associated with the process of vasculogenesis (new vessel development) in adults is in many instances indicative of ongoing ischemia, and may thus be used for the diagnosis and prognosis of ischemia. The inventors first made this discovery after noticing that a large number of genes in Flk1+ cells of mouse were upregulated during vasculogenesis (new vessel development). Detailed trans-species verification, wherein the expression of these vasculogenesis-related genes and their expression products in the developing vascular tree in mice and zebrafish was scrutinized, indicated that these genes were upregulated during ischemia in adolescent mouse models. This linked the expression of these genes to the arterial disease. The inventors have therefore now discovered that diagnosis of ongoing ischemia, as well as other cardiovascular diseases, may occur by detecting the compensatory neovascularization process, and in particular through detecting a gene expression profile associated with activated EPCs. These genes and their products provide a compensatory system during ischemia and arterial repair leading to compensatory vasculogenesis and vascular repair. The linking of the expression of these genes to ischemia was further verified by analysis of the expression of individual genes (clones) and their gene products in subsets of circulating polymorphonuclear leukocytes (PML), endothelial progenitor cells (as part of the PML fraction) and serum or blood samples from patients suffering from stable ischemic coronary artery disease and acute coronary syndrome.

In a first aspect, the present invention now provides a method of diagnosis or prognosis of cardiovascular disease in a subject, said method comprising the steps of detecting the presence of activated endothelial progenitor cells (EPCs) in a sample of a circulation fluid of said subject.

In a preferred embodiment of such a method, said cardiovascular disease is associated with arterial damage or myocardial damage.

In an alternative preferred embodiment of such a method, said cardiovascular disease is associated with ischemia.

In a method of the present invention, the step of detecting activated EPCs suitably comprises the detection in said sample of an increase in the gene expression level in EPCs of at least one gene and even more preferably at least 2, 3, 4, 5, 10, 15, 20, 25, 30, 35, or all genes selected from the group consisting of ADORA1, ADORA2A, ADORA2B, ADORA3, AGTRL1 (APLNR), AMPH, APLN, CCBE1, CDC42, CGNL1, CREBBP, CRIP1, CRIP2, CRIP3, CYB5B, DLL4, DUSP5, EEA1, egr-1, ELK1, ELK3, ELK4 (SAP1), EP300, ERG1 (KCNH2), ETS1, ETS2, EXOC3L, FGD1, FGD2, FGD3, FGD4, FGD5, FLT1, FST, GATA6, GRRP1, HO-1 (HMOX1), HO-2 (HMOX2), IFNG, IL1A, IL1B, LAMA4, Lamb1-1, LGMN, MMP3, Nos2, PAI1, PHD1, PLVAP, RAB5a, RIN3, ROCK2, SOX18, SOX7, SRF, STAB1, STAB2, STUB1, TFEC, THBS1, THBS2, THBS3, THBS4, THBS5, THSD1, TNFAIP8, and XLKD1 (LYVE1), still preferably at least one gene and yet still more preferably at least 2, 3, 4, 5, 10, 15, 20, 25, 30 or all genes selected from the group consisting of ADORA2A, AGTRL1 (APLNR), APLN, CCBE1, CGNL1, CRIP2, CYB5B, DLL4, DUSP5, ELK3, ERG1 (KCNH2), ETS1, ETS2, EXOC3L, FGD5, GRRP1, HO-1 (HMOX1), HO-2 (HMOX2), LAMA4, Lamb1-1, LGMN, PLVAP, RIN3, ROCK2, SOX7, SOX18, STAB1, STAB2, STUB1, TFEC, THSD1, TNFAIP8, and XLKD1 (LYVE1). The increase in the gene expression level may be detected by any suitable method and may be directed towards detection of nucleic acids (e.g. mRNA) or proteins. Protein expression products of the above-referred genes may be excreted by the activated EPC and the gene expression level in EPCs may thus be detected by detection of a protein in whole blood, instead of in an EPC fraction thereof.

An important advantage of this method over the prior art methods (counting of the number of circulatory EPCs, or ergometric (exercise) testing) is that the present method is more sensitive and that the disease can be detected at an earlier stage.

Based on this, the present inventors have found that potential markers for arterial disease, ischemia and compensatory neovascularization may be found among the genes and the products of these genes upregulated during vasculogenesis in activated EPCs, and that these may be detected in blood, serum or cellular fractions of blood.

In another aspect, the present invention now provides a biomarker for diagnosis or prognosis of cardiovascular disease in a patient, said biomarker comprising the expression product of a gene the expression of which is regulated during vasculogenesis. Preferably, said biomarker comprises the expression product of a gene the expression of which is upregulated during vasculogenesis (pro-vasculogenic genes). In an alternative preferred embodiment, the biomarker is present in endothelial progenitor cells (EPCs) or polymorphonuclear leukocytes (PMNs) or whole blood.

In principle, the invention is based on the detection of activated EPCs in blood of a subject. Activated EPCs, as part of the normal pool of circulating EPCs, can best be identified by their specific genetic repertoire (gene expression profile). Since some of the gene expression products are excreted by the cells in the surrounding blood, the excreted biomarker may be detected in whole blood as well.

In yet another preferred embodiment, said EPCs or PMNs are present in the peripheral blood of patients. Preferably, said EPCs are Flk1⁺ (Flk1 positive) cells. Most preferably, the activated EPCs display a gene expression profile wherein at least one gene and even more preferably at least 2, 3, 4, 5, 10, 15, 20, 25, 30, 35, or all genes selected from the group consisting of ADORA1, ADORA2A, ADORA2B, ADORA3, AGTRL1 (APLNR), AMPH, APLN, CCBE1, CDC42, CGNL1, CREBBP, CRIP1, CRIP2, CRIP3, CYB5B, DLL4, DUSP5, EEA1, egr-1, ELK1, ELK3, ELK4 (SAP1), EP300, ERG1 (KCNH2), ETS1, ETS2, EXOC3L, FGD1, FGD2, FGD3, FGD4, FGD5, FLT1, FST, GATA6, GRRP1, HO-1 (HMOX1), HO-2 (HMOX2), IFNG, IL1A, IL1B, LAMA4, Lamb1-1, LGMN, MMP3, Nos2, PAI1, PHD1, PLVAP, RAB5a, RIN3, ROCK2, SOX18, SOX7, SRF, STAB1, STAB2, STUB1, TFEC, THBS1, THBS2, THBS3, THBS4, THBS5, THSD1, TNFAIP8, and XLKD1 (LYVE1), still preferably at least one gene and yet still more preferably at least 2, 3, 4, 5, 10, 15, 20, 25, 30 or all genes selected from the group consisting of ADORA2A, AGTRL1 (APLNR), APLN, CCBE1, CGNL1, CRIP2, CYB5B, DLL4, DUSP5, ELK3, ERG1 (KCNH2), ETS1, ETS2, EXOC3L, FGD5, GRRP1, HO-1 (HMOX1), HO-2 (HMOX2), LAMA4, Lamb1-1, LGMN, PLVAP, RIN3, ROCK2, SOX7, SOX18, STAB1, STAB2, STUB1, TFEC, THSD1, TNFAIP8, and XLKD1 (LYVE1) is upregulated when compared to its expression level in non-activated EPCs.

A biomarker of the present invention is preferably an expression product (polypeptide or polyribonucleotide) of at least one gene and even more preferably at least 2, 3, 4, 5, 10, 15, 20, 25, 30, 35, or all genes selected from the group consisting of ADORA1, ADORA2A, ADORA2B, ADORA3, AGTRL1 (APLNR), AMPH, APLN, CCBE1, CDC42, CGNL1, CREBBP, CRIP1, CRIP2, CRIP3, CYB5B, DLL4, DUSP5, EEA1, egr-1, ELK1, ELK3, ELK4 (SAP1), EP300, ERG1 (KCNH2), ETS1, ETS2, EXOC3L, FGD1, FGD2, FGD3, FGD4, FGD5, FLT1, FST, GATA6, GRRP1, HO-1 (HMOX1), HO-2 (HMOX2), IFNG, IL1A, IL1B, LAMA4, Lamb1-1, LGMN, MMP3, Nos2, PAI1, PHD1, PLVAP, RAB5a, RIN3, ROCK2, SOX18, SOX7, SRF, STAB1, STAB2, STUB1, TFEC, THBS1, THBS2, THBS3, THBS4, THBS5, THSD1, TNFAIP8, and XLKD1 (LYVE1), still preferably at least one gene and yet still more preferably at least 2, 3, 4, 5, 10, 15, 20, 25, 30 or all genes selected from the group consisting of ADORA2A, AGTRL1 (APLNR), APLN, CCBE1, CGNL1, CRIP2, CYB5B, DLL4, DUSP5, ELK3, ERG1 (KCNH2), ETS1, ETS2, EXOC3L, FGD5, GRRP1, HO-1 (HMOX1), HO-2 (HMOX2), LAMA4, Lamb1-1, LGMN, PLVAP, RIN3, ROCK2, SOX7, SOX18, STAB1, STAB2, STUB1, TFEC, THSD1, TNFAIP8, and XLKD1 (LYVE1).

The biomarker of the invention may have the form of the expression product of one of the genes referred to above, or may take the form of a protein profile or RNA profile.

In another aspect, the present invention provides the use of a biomarker as defined above for the diagnosis or prognosis of cardiovascular disease in a subject.

In another aspect, the present invention provides the use of a biomarker as defined above as a surrogate end-point marker. Such a surrogate end-point marker may be used in prognostic studies into cardiovascular disease and for testing efficacy of therapy.

In another aspect, the present invention provides a method for the diagnosis or prognosis of cardiovascular disease in a subject, comprising detecting in the blood of said subject a biomarker according to present invention. Preferably said method is performed on a sample of blood of said subject. In other preferred embodiments of said method, the step of detecting the biomarker is performed by using microarrays. In alternative preferred embodiments of said method, the step of detecting the biomarker is performed by using tandem mass spectrometry (MS-MS), by MALDI-FT mass spectrometry, MALDI-FT-ICR mass spectrometry, MALDI Triple-quad mass spectrometry, QPCR or other hybridisation method or immunoassay. In fact, any suitable detection method can be used to identify the biomarker RNA or protein.

In another aspect, the present invention provides a kit-of-parts for performing a method for the diagnosis or prognosis of cardiovascular disease in a subject according to the present invention. Said kit comprises at least one biomarker as defined herein above, or a specific binding partner that binds specifically to said biomarker. A kit according to the present invention optionally further comprising one or more of the following:
at least one reference or control sample;
information on the reference value for the biomarker;
at least one test compound capable of binding to said specific binding partner;
at least one detectable marker for detecting binding between said biomarker and said specific binding partner.

In another aspect, the present invention provides a microarray for performing a method for the diagnosis or prognosis of cardiovascular disease in a subject according to the present invention. Said microarray comprises specific binding partners that bind specifically to at least two biomarkers as defined herein above bound to a solid support.

In another aspect, the present invention provides a diagnostic reagent that binds specifically to a biomarker as defined herein above. Preferably, the diagnostic reagent is an antibody or a nucleic acid molecule specifically hybridizing under stringent conditions to said biomarker.

In another aspect, the present invention provides a diagnostic composition comprising a diagnostic reagent of the present invention.

In another aspect, the present invention provides the use of a diagnostic composition of the present invention, for diagnosing cardiovascular disease in a subject.

In another aspect, the present invention provides a method of treating a subject (having an increased risk of) suffering from cardiovascular disease, said method comprising using a biomarker as defined herein above as a therapeutic target or as a therapeutic agent.

In a method of treating a subject according to the present invention said use of said biomarker as a therapeutic target or as a therapeutic agent comprises affecting the expression at least one gene and even more preferably at least 2, 3, 4, 5, 10, 15, 20, 25, 30, 35, or all genes selected from the group consisting of ADORA1, ADORA2A, ADORA2B, ADORA3, AGTRL1 (APLNR), AMPH, APLN, CCBE1, CDC42, CGNL1, CREBBP, CRIP1, CRIP2, CRIP3, CYB5B, DLL4, DUSP5, EEA1, egr-1, ELK1, ELK3, ELK4 (SAP1), EP300, ERG1 (KCNH2), ETS1, ETS2, EXOC3L, FGD1, FGD2, FGD3, FGD4, FGD5, FLT1, FST, GATA6, GRRP1, HO-1 (HMOX1), HO-2 (HMOX2), IFNG, IL1A, IL1B, LAMA4, Lamb1-1, LGMN, MMP3, Nos2, PAI1, PHD1, PLVAP, RAB5a, RIN3, ROCK2, SOX18, SOX7, SRF, STAB1, STAB2, STUB1, TFEC, THBS1, THBS2, THBS3, THBS4, THBS5, THSD1, TNFAIP8, and XLKD1 (LYVE1), still preferably at least one gene and yet still more preferably at least 2, 3, 4, 5, 10, 15, 20, 25, 30 or all genes selected from the group consisting of ADORA2A, AGTRL1 (APLNR), APLN, CCBE1, CGNL1, CRIP2, CYB5B, DLL4, DUSP5, ELK3, ERG1 (KCNH2), ETS1, ETS2, EXOC3L, FGD5, GRRP1, HO-1 (HMOX1), HO-2 (HMOX2), LAMA4, Lamb1-1, LGMN, PLVAP, RIN3, ROCK2, SOX7, SOX18, STAB1, STAB2, STUB1, TFEC, THSD1, TNFAIP8, and XLKD1 (LYVE1).

Preferably, said use of said biomarker as a therapeutic target comprises decreasing the amount of at least one protein that is over-expressed in subjects (having an increased risk of) suffering from cardiovascular disease, or increasing the amount of at least one protein that is under-expressed in subjects (having an increased risk of) suffering from cardiovascular disease.

In another aspect, the present invention provides a pharmaceutical composition for the treatment of an increased risk of suffering a cardiovascular event, comprising at least one inhibitor compound selected from:
an antibody or derivative thereof directed against the biomarker of the present invention, preferably a biomarker expressed on the cell membrane, and said derivative preferably being selected from the group consisting of scFv fragments, Fab fragments, chimeric antibodies, bifunctional antibodies, intrabodies, and other antibody-derived molecules;
a biomarker as defined herein;
a small molecule interfering with the biological activity of said biomarker;
an antisense molecule, in particular an antisense RNA or antisense oligodeoxynucleotide;
an RNAi molecule and
a ribozyme,
   and a suitable excipient, carrier or diluent.

In another aspect, the present invention provides a method of treating a subject, comprising administering to said subject the pharmaceutical composition of the present invention in an amount effective to decrease (the risk of) cardiovascular disease.

### DETAILED DESCRIPTION OF THE INVENTION

### Terminology

The term “endothelial progenitor cell (EPC)” refers to a circulating, bone marrow-derived cell population that appears to participate in both vasculogenesis and vascular homeostasis. This progenitor (stem) cell population were first described as CD34⁺ CD133⁺ cells in the bone marrow by Asahara et al. in 1997 (Science Vol. 275, 964-967), but can be isolated from the peripheral blood mononuclear cell (PBMC) fraction of blood. Seen in small numbers in the blood of healthy individuals, their numbers tend to increase following vascular injury. So far, experiments have established the ability of EPC to form colonies in vitro, suggesting a role in both angiogenesis and in the maintenance of existing vessel walls. Recent evidence has suggested the involvement of EPC in tumor vasculogenesis.

The term “activated endothelial progenitor cells (EPCs)” refers to EPCs having a gene expression profile that differs from normal circulating EPC. This gene expression profile may for instance be recognized by virtue of the upregulation of the expression of the genes of Table 1. However, since the genes in Table 1 are indicated as biomarkers which may be detected in blood, these only include genes that are upregulated, and thus result in a positive expression of a product. The person of average skill in the art will recognize that down-regulated genes may also be observed in activated EPCs, but that such genes are not suitable for use as biomarkers. However, such down-regulated genes may be used as genes part of an expression profile that is indicative of an activated EPC. Whether an EPC is an activated EPC is thus best assessed by assessing the expression profile of an EPC and comparing that profile to the specific profile as disclosed herein comprising an increased expression of the genes of Table 1, or by determining the expression level of one or more genes of Table 1 and determining whether the level is increased as compared to a control EPC (i.e. a circulating EPC in the blood of a normal healthy subject).

“Vasculogenesis” (also referred herein as neovascularisation or neoangiogenesis) is the formation of blood vessels when there are no pre-existing blood vessels, in contrast to angiogenesis, which term refers to the development of blood vessels from existing ones. Vasculogenesis was first believed to occur only during embryologic development, although is now known that the process also occurs in adult organisms. Vasculogenesis involves migration and differentiation of endothelial precursor cells (angioblasts) in response to local cues (such as growth factors and extracellular matrix) and the formation of new blood vessels (vasular trees). These vascular trees are then pruned and extended through angiogenesis. Circulating endothelial progenitor cells (derivatives of stem cells) are known to contribute, albeit to varying degrees, to neovascularization.

The term “during vasculogenesis” as used herein, refers to the period wherein gene expression is geared towards vasculogeneis, rather than angiogenesis. The formation of new blood vessels proceeds by both vasculogenesis and angiogenesis. During embryogenesis, the period of vasculogenesis is characterized by a peak in predominance of Flk1-positive embryonic stem cells. The mouse Flk1 gene encodes the major signaling receptor, vascular endothelial growth factor receptor 2 (VEGFR-2), for vascular endothelial growth factor A (VEGF-A), and is essential for development of the vascular and hematopoietic systems in the early embryo. In mice, mouse embryonic stem (ES) cells differentiate into Flk1+ cells, which give rise to two types of cells, i.e. mural cells (vascular smooth muscle cells identified by (but not exclusively) expression of α-smooth muscle actin; SMA+) and endothelial cells (identified by (but not exclusively) expression of platelet-endothelial cell adhesion molecule; PECAM1+). These mural cells and endothelial cells subsequently assemble into primitive blood vessels. Thus, Flk1-positive cells derived from embryonic stem cells serve as vascular progenitors.

Vasculogenesis can be differentiated from angiogenesis as follows. Vasculogenesis is the de novo synthesis of blood vessels from stem cells (progenitor cells) and involves recruitment and differentiation of these pleitrophic cells, whereas angiogenesis is the formation of new vessels from existing ones (dedifferentiation of endothelial cells, migration/proliferation and again differentiation into new tubules and remodelling into hemodynamic significant vessels (“pruning”).

The term “ischemic cardiovascular event” or short “ischemic event”, as used herein refers to an interruption of the blood supply to an organ or tissue. An ischemic event may often be the result of a blood cloth and in patients with atherosclerotic stenosis is most often caused when emboli dislodge from the atherosclerotic lesion. The resulting stenosis, or narrowing or blockage of an artery or other vessel due to this obstruction may result in a large number of adverse conditions, many of which have severe consequences for the subject. Ischemic cardiovascular events as referred to herein include, but are not limited to stroke/transient ischemic attack or cerebrovascular attack, myocardial infarction, myocardial ischemia (angina pectoris), any cardiomyopathy complicated by myocardial ischmia (for instance symptomatic aortic stenosis, HOCM), cerebral bleeding, peripheral (unstable) angina pectoris, claudicatio intermittens (peripheral atherosclerotic artery disease) and other major abnormalities occurring in the blood vessels. The term “abnormalities occurring in the blood vessels” includes reference to coronary and cerebrovascular events as well as to peripheral vascular disease. The term “ischemic cardiovascular event” is often the acute stage of a medical condition that is broadly encompassed by the term “cardiovascular disease”.

The term “ischemia”, as used herein, refers to an absolute or relative shortage of the blood supply or an inadequate flow of blood to an organ, body part or tissue. Relative shortage refers to the discrepancy between blood supply (oxygen delivery) and blood request (oxygen consumption by tissue). The restriction in blood supply, generally due to factors in the blood vessels, is most often, but not exclusively, caused by constriction or blockage of the blood vessels by thromboembolism (blood clots) or atherosclerosis (lipid-laden plaques obstructing the lumen of arteries). Ischemia result in damage or dysfunction of tissue. Ischemia of the heart muscle results in angina pectoris, and is herein referred to as ischemic heart disease.

The term “cardiovascular disease” (CVD) generally refers to a number of diseases that affect the heart and circulatory system, including aneurysms; angina; arrhythmia; atherosclerosis; cardiomyopathies; cerebrovascular accident (stroke); cerebrovascular disease; congenital heart disease; congestive heart failure; coronary heart disease (CHD), also referred to as coronary artery disease (CAD), ischemic heart disease or atherosclerotic heart disease; dilated cardiomyopathy; diastolic dysfunction; endocarditis; heart failure; hypertension (high blood pressure); hypertrophic cardiomyopathy; mitral valve prolapse; myocardial infarction (heart attack); myocarditis; peripheral vascular disease; rheumatic heart disease; valve disease; and venous thromboembolism. As used herein, the term “cardiovascular disease” also encompasses reference to ischemia; arterial damage (damage to the endothelial lineage) due to physical damage (endartiectomie, balloon angioplasty) or as a result of chronic damage (including atherosclerosis); myocardial damage (myocardial necrosis); and myonecrosis. In general, any physiological or pathophysiological condition that elicits a neoangiogenic response is encompassed by the term “cardiovascular disease” as used herein.

The term “circulatory fluid” refers to both lymphatic fluid and blood, preferably blood.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide”, “peptide” and “protein” include glycoproteins and proteins comprising any other modification, as well as non-glycoproteins and proteins that are otherwise unmodified.

“Protein profile”, as used herein, refers to the collection of proteins, protein fragments, or peptides present in a sample. The protein profile may comprise the identities (e.g., specific names or amino acid sequence identities of known proteins, or molecular weights or other descriptive information about proteins that have not been further characterized) of the proteins in a collection, without reference to quantity present. In other embodiments, a protein profile includes quantitative information for the proteins represented in a sample. In analogy, “gene expression profile” as used herein, refers to the collection of gene expression products (including such products as proteins and RNA molecules) present in a sample.

“Quantitation”, as used herein with reference to expression products in a gene expression profile refers to the determination of the amount of a particular protein, peptide or RNA present in a sample. Quantitation can be either in absolute amount (e.g., μg/ml) or a relative amount (e.g., relative intensity of signals). Usually such procedures are performed by dedicated biochemical assays, such chromatographic, mass spectrometric or hybridization assays. “Quantitation”, as used herein with reference to cells in a circulatory fluid refers to the determination of an absolute or relative number of cells. Usually such procedures are performed by dedicated cell counters, such as flow cytometers.

“Marker” and “Biomarker” are used interchangeably to refer to a gene expression product that is differentially present in a samples taken from two different subjects, e.g., from a test subject or patient having (a risk of developing) an ischemic event, compared to a comparable sample taken from a control subject (e.g., a subject not having (a risk of developing) an ischemic event; a normal or healthy subject). Alternatively, the terms refer to a gene expression product that is differentially present in a population of cells relative to another population of cells.

The phrase “differentially present” refers to differences in the quantity or frequency (incidence of occurrence) of a marker present in a sample taken from a test subject as compared to a control subject. For example, a marker can be a gene expression product that is present at an elevated level or at a decreased level in blood samples of a risk subjects compared to samples from control subjects. Alternatively, a marker can be a gene expression product that is detected at a higher frequency or at a lower frequency in samples of blood from risk subjects compared to samples from control subjects.

A gene expression product is “differentially present” between two samples if the amount of the gene expression product in one sample is statistically significantly different from the amount of the gene expression product in the other sample. For example, a gene expression product is differentially present between two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

As used herein, the terms “antibody” and “antibodies” refer to monoclonal antibodies, multispecific antibodies, synthetic antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies of the present invention include immunoglobulin molecules and immunologically active portions of immunoglobulin molecules, i.e., molecules that contain an antigen binding site that immunospecifically binds to a polypeptide antigen encoded by a gene comprised in the genomic regions or affected by genetic transformations in the genomic regions listed in Table 1. The immunoglobulin molecules of the invention can be of any type (e.g., IgG, IgE, IgM, IgD, IgA and IgY), class (e.g., IgG₁, IgG₂, IgG₃, IgG₄, IgA₄ and IgA₂) or subclass of immunoglobulin molecule.

“Immunoassay” is an assay that uses an antibody to specifically bind an antigen (e.g., a marker). The immunoassay is characterized by the use of specific binding properties of a particular antibody to isolate, target, and/or quantify the antigen. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

The phrase “specifically (or selectively) binds” when referring to an antibody, or “specifically (or selectively) immunoreactive with”, when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein.

The terms “affecting the expression” and “modulating the expression” of a protein or gene, as used herein, should be understood as regulating, controlling, blocking, inhibiting, stimulating, enhancing, activating, mimicking, bypassing, correcting, removing, and/or substituting said expression, in more general terms, intervening in said expression, for instance by affecting the expression of a gene encoding that protein.

The terms “subject” or “patient” are used interchangeably herein and include, but are not limited to, an organism; a mammal, including, e.g., a human, non-human primate, mouse, pig, cow, goat, cat, rabbit, rat, guinea pig, hamster, horse, monkey, sheep, or other non-human mammal; and a non-mammal, including, e.g., a non-mammalian vertebrate, such as a bird (e.g., a chicken or duck), an amphibian and a fish, and a non-mammalian invertebrate.

### Description of the Preferred Embodiments

### Biomarkers

In 1999, Asahara first described that the endothelial progenitor cell (EPC) in the peripheral blood of patients constitutes a pool of recruited endothelial precursor cells that respond to ischemia and arterial damage. Since then, these cells have been shown to be involved in neoangiogenesis (new vessel development) under physiological and pathophysiological conditions. Moreover, EPCs are involved in the ongoing arterial repair and/or regeneration following damage to the endothelial lineage not only by physical damage (endartiectomie, balloon angioplasty), but also by chronic damage (including atherosclerosis) and ischemia/myonecrosis (eliciting a neoangiogenesis response).

Despite this knowledge, there has hitherto not been provided a biomarker for the diagnosis or prognosis of cardiovascular disease. Markers for myocardial damage (myocardial necrosis markers) are routinely used in the cardiological practice but mainly comprise the identification of intracellular myocardial enzymes, that are released in the circulation following damage to the myocardial tissue, including troponin and creatinin kinase MB subfraction. However, no markers exist as of to date that can quantify ongoing ischemia or previous ischemic events. Stable angina constitutes the majority of the cardiovascular practice and comprises a considerable patient population in western society (by shear volume of morbidity and mortality). Alternative diagnostic methods, including exercise testing and perfusion imaging are not cost effective and lack proper sensitivity and specificity. This serious health threat warrants proper biomarkers to identify patients at risk and evaluate proper response to anti ischemic therapy.

In search for such markers, the present inventors have performed genome wide analysis (using RNA microarray analysis) of embryonic vessel development in mouse and zebrafish and identified over 2000 genes that are in some way involved in arterial development. This large number of genes was initially found after determining the RNA expression profile of Flk1⁺ embryonic stem cells in 8-11 days old mouse embryo's, using differential expression between the Flk1+ embryonic stem cell versus a Flk− (minus) population (non-relevant cells). By using whole-mount in situ hybridization (WISH), 1150 genes were selected as providing potential biomarkers. The inventors further selected and verified expression of these candidate genes in the developing vascular tree in mice and zebrafish, and their upregulation during ischemia in adolescent mouse models.

Of the 2000+ cells the present inventors identified 26 clones involved in human, murine, and zebrafish vasculogenesis based on orthology search. Flk1+ cells designate both early hematopoietic stem cells, dedicated angioblasts, as well as fully differentiated endothelial cells, thereby encompassing the full differentiation process from hemangioblasts to endothelial cell (EC). It is known that that Flk1 and Tal1 are two of the first early markers on dedicated angioblasts during early development, whereas Flk1 expression is rapidly downregulated in extra-embryonic hematopoietic stem cells, as they commit to the hematopoietic lineage. Using in situ hybridization studies in the zebrafish the inventors were initially able to show that 23% of the genes, identified by differential display analysis comparing Flk1+ vs Flk1− cells, were exclusively expressed at sites of vasculogenesis, with another 30% showed expression both at sites of vasculogenesis as well as neuronal and retinal epitheloid tissue. This emphasized the validity of the original experimental rationale of the inventors to identify genetic regulators of vasculogenesis by this particular gene screen. The inventors identified 2000+ murine genes differentially expressed during mouse development in the vascular tree and performed high throughput whole mount in situ hybridization of vasculogenesis during zebrafish development, as well as quantitative PCR analysis of selected genes using various tissues collected from murine models of ischemia and human disease to verify proper spatiotemperal expression in the developing vascular bed during zebra fish development. Using this screen for genes involved in different manifestations of vasculogenesis in mice, zebrafish and humans, the inventors were able to identify common regulatory gene products preserved throughout species and different models, and were able to identify common genetic regulators of vasculogenesis in the embryonic and adult mouse.

The role of vasculogenesis in adult neovessel formation is well established and has been the subject of numerous scientific papers. Yet, the genetic regulation of the process remains unclear to date. The present inventors have studied and compared vasculogenesis during mouse and zebrafish development as a model to analyze in vivo the process of vasculogenesis in the absence of hematopoiesis and angiogenesis. Subsequently the inventors have cross-correlated the expression of the clones that were identified with expression in (adult) mouse models of limb ischemia and human disease. By doing that, the inventors were able to identify clones expressed both during embryonic and adult vasculogenesis. These clones have been further studied in vivo in the (adult) mouse model of hind limb ischemia and by use of (morpholino) knock down analysis in the developing zebra fish. Using these technologies the inventors were able to identify 26 candidate regulatory genes involved in the adult and embryonic vasculogenesis.

Although it remains unclear whether adult and embryonic vasculogenesis is regulated by common pathways, the inventors were able to identify shared expression patterns, possibly identifying shared genetic regulators.

Finally, induced expression of individual clones was verified by QPCR analysis in subsets of circulating PML of blood samples obtained from patients admitted with stable ischemic coronary artery disease and with acute coronary syndrome.

Based on findings obtained through these studies, the inventors have gained an imperative insight in the molecular mechanisms of vasculogenesis and angioblast differentiation in ischemic disease and arterial repair and identified a genetic repertoire or gene expression profile that is characteristic by genes involved in EPC recruitment, activation and migration into areas of neovascularization due to ischemia and arterial injury, and which can be used as indicators of the presence of activated EPCs as a specific EPC phenotype, and which can thus be used as indicators of ongoning vasculogenesis and arterial repair following ischemia and arterial injury in a broad setting, in particular those cardiovascular diseases associated with arterial damage, myocardial damage or ischemia.

A total of 26 genes were found that constituted the activated EPC phenotype, and that proved of value as a biomarker for these disorders. The skilled person will immediately understand that these genes are suitable not only as biomarkers for the above-referred pathologies, but also as biomarkers for the physiological process of vasculogenesis, preferably in adult subjects, and that these genes may be used as therapeutic targets for treating these pathologies, or for stimulating the physiological process of vasculogenesis. The genes are listed in Table 1.

TABLE 1
List of 33 genes of which the expression is upregulated during
ischemic heart disease. It is to be understood that homologs in for other species are
included herein.

| Official Symbol | Full Name | GenBank GeneID ^{a} |
|---|---|---|
| ADORA2A | adenosine A2a receptor | Homo sapiens GeneID: 135 |
| AGTRL1 | angiotensin II receptor-like 1 | Homo sapiens GeneID: 187 |
| (APLNR) | | |
| APLN | apelin, AGTRL1 ligand | Homo sapiens GeneID: 8862 |
| CCBE1 | collagen and calcium binding EGF domains 1 | Homo sapiens GeneID: 147372 |
| CGNL1 | cingulin-like 1 | Homo sapiens GeneID: 84952 |
| CRIP2 | cysteine-rich protein 2 | Homo sapiens GeneID: 1397 |
| CYB5B | cytochrome b5 type B (outer mitochondrial membrane) | Homo sapiens GeneID: 80777 |
| DLL4 | delta-like 4 (Drosophila) | Homo sapiens GeneID: 54567 |
| DUSP5 | dual specificity phosphatase 5 | Homo sapiens GeneID: 1847 |
| ELK3 | ELK3, ETS-domain protein (SRF accessory protein 2) | Homo sapiens GeneID: 2004 |
| ERG1 (KCNH2) | potassium voltage-gated channel, subfamily H (eag-related), | Homo sapiens GeneID: 3757 |
| | member 2 | |
| ETS1 | v-ets erythroblastosis virus E26 oncogene homolog 1 (avian) | Homo sapiens GeneID: 2113 |
| ETS2 | v-ets erythroblastosis virus E26 oncogene homolog 2 (avian) | Homo sapiens GeneID: 2114 |
| EXOC3L | exocyst complex component 3-like | Homo sapiens GeneID: 283849 |
| FGD5 | FYVE, RhoGEF and PH domain containing 5 | Homo sapiens GeneID: 152273 |
| GRRP1 | glycine/arginine rich protein 1 | Homo sapiens GeneID: 79927 |
| HO-1 (HMOX1) | heme oxygenase (decycling) 1 | Homo sapiens GeneID: 3162 |
| HO-2 (HMOX2) | heme oxygenase (decycling) 2 | Homo sapiens GeneID: 3163 |
| LAMA4 | laminin, alpha 4 | Homo sapiens GeneID: 3910 |
| Lamb1-1 | laminin B1 subunit 1 | Mus musculus GeneID: 16777 |
| LGMN | Legumain | Homo sapiens GeneID: 5641 |
| PLVAP | plasmalemma vesicle associated protein | Homo sapiens GeneID: 83483 |
| RIN3 | Ras and Rab interactor 3 | Homo sapiens GeneID: 79890 |
| ROCK2 | Rho-associated, coiled-coil containing protein kinase 2 | Homo sapiens GeneID: 9475 |
| SOX7 | SRY (sex determining region Y)-box 7 | Homo sapiens GeneID: 83595 |
| SOX18 | SRY (sex determining region Y)-box 18 | Homo sapiens GeneID: 54345 |
| STAB1 | stabilin 1 | Homo sapiens GeneID: 23166 |
| STAB2 | stabilin 2 | Homo sapiens GeneID: 55576 |
| STUB1 | STIP1 homology and U-box containing protein 1 | Homo sapiens GeneID: 10273 |
| TFEC | transcription factor EC | Homo sapiens GeneID: 22797 |
| THSD1 | thrombospondin, type I, domain containing 1 | Homo sapiens GeneID: 55901 |
| TNFAIP8 | TNFalpha inducible protein 8 | Homo sapiens GeneID: 25816 |
| XLKD1 (LYVE1) | extracellular link domain containing 1 (lymphatic vessel | Homo sapiens GeneID: 10894 |
| | endothelial hyaluronan receptor 1) | |

^{a} Maglott et al. Entrez Gene: gene-centered information at NCBI. Nucleic Acids Research,
2006, Vol. 00, Database issue D1-D6

In addition to the above 26 genes of which the expression is upregulated during ischemia, the skilled person will readily understand that genes, or the expression products thereof, that interact with these 26 genes, or with the expression products thereof, are also indicated as candidate biomarkers in aspects of the present invention. Such interaction may include protein-protein interaction, protein-DNA interaction, RNA-DNA interaction, receptor ligand interaction, or any type of interaction encountered under normal physiological conditions. Alternatively, or in addition, the skilled person will readily understand that genes or the expression products thereof, that are members of the same gene family to which one of these 26 genes belongs, are also encompassed herein as candidate biomarkers in aspects of the present invention. The rationale for this is that an expression pattern mostly involves cascades of interacting genes and/or genes of the same family. Hence, Crip family genes other than Crip 2 are, for instance, also aspects of the present invention. These interacting or associated genes are indicated in Table 2.

TABLE 2
Genes that interact with or are family members of the genes of which the
expression is upregulated during ischemic heart disease.

| Official Symbol | Full Name | GenBank GeneID |
|---|---|---|
| ADORA1 | adenosine A1 receptor | Homo sapiens GeneID: 134 |
| ADORA2B | adenosine A2b receptor | Homo sapiens GeneID: 136 |
| ADORA3 | adenosine A3 receptor | Homo sapiens GeneID: 140 |
| AMPH | Amphiphysin | Homo sapiens GeneID: 273 |
| CDC42 | cell division cycle 42 (GTP binding protein, 25kDa) | Homo sapiens GeneID: 998 |
| CREBBP | CREB Binding Protein | Homo sapiens GeneID: 1387 |
| CRIP1 | cysteine-rich protein 1 (intestinal) | Homo sapiens GeneID: 1396 |
| CRIP3 | cysteine-rich protein 3 | Homo sapiens GeneID: 401262 |
| EEA1 | Early endosome antigen 1 | Homo sapiens GeneID: 8411 |
| egr-1 | early growth response 1 | Mus musculus GeneID: 13653 |
| ELK1 | ELK1, member of ETS oncogene family | Homo sapiens GeneID: 2002 |
| ELK4 (SAP1) | ELK4, ETS-domain protein (SRF accessory protein 1) | Homo sapiens GeneID: 2005 |
| EP300 | E1A binding protein p300 | Homo sapiens GeneID: 2033 |
| FLT1 | fms-related tyrosine kinase | Homo sapiens GeneID: 2321 |
| FGD1 | FYVE, RhoGEF and PH domain containing 1 | Homo sapiens GeneID: 2245 |
| FGD2 | FYVE, RhoGEF and PH domain containing 2 | Homo sapiens GeneID: 221472 |
| FGD3 | FYVE, RhoGEF and PH domain containing 3 | Homo sapiens GeneID: 89846 |
| FGD4 | FYVE, RhoGEF and PH domain containing 4 | Homo sapiens GeneID: 121512 |
| FST | Follistatin | Homo sapiens GeneID: 10468 |
| GATA6 | GATA binding protein 6 | Homo sapiens GeneID: 2627 |
| IFNG | interferon, gamma | Homo sapiens GeneID: 3458 |
| IL1A | interleukin 1, alpha | Homo sapiens GeneID: 3552 |
| IL1B | interleukin 1, beta | Homo sapiens GeneID: 3553 |
| MMP3 | matrix metallopeptidase 3 (stromelysin 1, progelatinase) | Homo sapiens GeneID: 4314 |
| Nos2 | nitric oxide synthase 2, inducible, macrophage | Mus musculus GeneID: 18126 |
| PAI1 | nexin, plasminogen activator inhibitor type 1, member 1 | Homo sapiens GeneID: 5054 |
| PHD1 | egl nine homolog 2 (C. elegans) | Homo sapiens GeneID: 112398 |
| RAB5a | RAB5A, member RAS oncogene family | Homo sapiens GeneID: 5868 |
| SRF | serum response factor | Homo sapiens GeneID: 6722 |
| THBS1 | thrombospondin 1 | Homo sapiens GeneID: 7057 |
| THBS2 | thrombospondin 2 | Homo sapiens GeneID: 7058 |
| THBS3 | thrombospondin 3 | Homo sapiens GeneID: 7059 |
| THBS4 | thrombospondin 4 | Homo sapiens GeneID: 7060 |
| THBS5 | thrombospondin 5 | Homo sapiens GeneID: 1311 |

The amount of expression products (RNA or protein) of these genes in cells provides insight in the level of expression of these genes. The skilled person is well aware of the various techniques available for studying the level of expression of genes in cells and tissue.

The biomarker may relate to the expression product of one of the genes listed in table 1, or may relate to the expression product of two, three, four or more genes listed in table 1. When the level of expression of multiple genes listed in table 1 is determined, an expression profile may be obtained that provides statistically very reliable correlation with ischemic heart disease.

This pro-vasculogenic profile, which provides a vasculogenic signature that indicates activation of the vascular repair response to ischemia and arterial damage, is of great importance to cardiovascular medical practice on the level of diagnosis, prognosis (e.g. for use as surrogate end point marker), and therapy.

The pro-vasculogenic gene expression profile composed of a set of individual biomarkers, may be used as a biomarker itself.

The biomarkers can be used to identify patients that lack a proper/adequate response to treatment of ischemic events. This could be very helpful to stratify patients to a high or low risk profile, that may be prone to develop additional ischemic events or inadvertent events in the future or may develop an improper vascular response to coronary intervention (PCI) leading up to restenosis or in-stent thrombosis or either predict a sub-optimal response to percutaneous intervention (for instance high risk for restenosis formation due to improper/inadequate vascular/endothelial repair response (=vasculogenesis)). This is helpful in determining further medical intervention by intensified medical monitoring, more aggressive revascularization strategies or individually tailored pharmacotherapy, including, but not limited to, continued dual antiplatelet therapy to prevent in-stent thrombosis.

The biomarkers of the invention may be used as surrogate end point markers. A surrogate end point marker is a biomarker intended to substitute for a clinical endpoint or intended to be used to delineate therapy efficacy (for instance anti ischemic therapy). In many settings, the primary clinical endpoint takes large, long term trials, which are, obviously expensive. Evaluation of real or hard end points in medical trials, including death, myocardial infarction or stroke would require study of a large study population which would be financially and from a moral/ethical point of view undesirable. Rather than evaluating hard end points in medical trials, surrogate end points are used as alternative indicators/predictors of improved outcome (and survival of the cardiovascular patient), including for instance global left ventricular function, or BNP analysis as a predictor for heart failure. Thus, the use of surrogate endpoints can also potentially prevent otherwise undesired endpoints, such as death. Surrogate end point markers are of eminent importance to testing the efficacy of medication. Ischemic heart disease lacks a proper biomarker as a surrogate end point marker to evaluate and predict proper response to therapy and therefore prognosis of the individual patients. The pro-vasculogenic biomarkers of the present invention may serve as biomarkers for medical treatment surrogate endpoint marker.

One may evaluate these markers as predictors of prognosis, but also their response to anti-ischemic therapy or therapy aimed to stimulate the vasculogenic response. Therefore the expression of these newly identified vasculogenic biomarkers may help to evaluate the effect of pro-vasculogenic pharmacotherapy, for instance therapies involving treatment with Granulocyte Colony-Stimulating Factor (GCSF), statins, erythropoietin, estrogens or exercise.

Biomarkers can serve as a method to determine patient prognosis, as they predict the proper response to an ischemic event and the initiation of a compensatory vascular development in the ischemic area. Alternatively, the analysis of pro-vasculogenic markers can evaluate the proper response to the initiated medical intervention (by pharmacotherapy of other intervention) in the individual patient. Hence the response to therapy can be evaluated in an early phase after therapy and individually adjusted, rather than an empirical approach on clinical grounds (wait-and-see approach). This could lead to a more individually tailored pharmacotherapy of the cardiovascular patient that allows adaptation to the medical strategy.

The biomarkers of the invention may be measured by any available method. A very suitable method is the use of customized chiparrays (DNA microarrays) capable of specifically hybridizing under stringent conditions to the gene expression products (RNAs) of the biomarkers of the present invention. The measurements provide a biomarker profile. These chiparrays may be used to test patient populations having cardiovascular disease for the presence of the biomarker profile indicative of the vasculogenic response as described herein that occurs during mammalian (human and murine) and amphibian embryogenesis and ischemia.

The biomarkers or the biomarker profile of the present invention represent a valuable tool for diagnosis and evaluation, as well as staging of cardiovascular patients. Such tools are currently unavailable. Second, these vasculogenic biomarkers constitute a novel therapeutic intervention for these patients or may be used to evaluate the response of patients to initiated therapy, thereby making medical decision making more effective.

The impact of the biomarkers of the present invention on daily clinical practice of the cardiovascular practice and the practice of the general practitioner is far-reaching since interpretation of surrogate endpoint biomarkers is much more unequivocal. This will eventually result in a considerable cost reduction and improved (and optimized) medical care for cardiovascular patients.

### Prognostic and Diagnostic Methods

In a method of the present invention for predicting the risk of a subject developing an ischemic events, the biomarker may be detected in a subject by in vivo or non-invasive methods or by ex vivo methods involving the removal of sample from the patient. “Detect” refers to identifying the presence, absence or amount of the object to be detected. Detection may comprise the demonstration of the presence, in absolute (e.g., μg/ml) terms or in relative terms (e.g., relative intensity of signals), or of the absence of the biomarker in (a sample of) a subject. Very suitable, the amount of the biomarker relative to another protein stably present in the subject, such as a household enzyme, may be determined in order to detect the biomarker in a subject.

A very suitable sample for detecting the biomarkers of the invention is a blood sample. In particular the biomarkers of the invention may be detected in polymorphonuclear leukocytes, endothelial progenitor cells in a blood sample or in whole blood (including serum).

Non-invasive methods for detecting or measuring proteins in the body of a subject (in vivo) are well known to the artisan. Such methods may include MRI, ultrasound spectroscopy, Raman spectroscopy and/or infra red spectroscopy and generally involve the use of specific labels for detection of the proteins.

Similar methods may be employed when analysing blood samples for the same purpose. However, in addition, ex vivo methods may be applied on samples that are obtained by invasive methods, and include the use of mass spectrometry and/or immunoassay analysis for detection and/or quantification of the proteins or RNA in blood samples. In addition, a large number of microarray techniques are available to detect or measure a large number of biomarkers simultaneously in a single assay. Such microarrays assays include DNA microarrays, such as cDNA microarrays and oligonucleotide microarrays; protein microarrays; and antibody microarrays.

A blood sample may be provided by removing a sample of blood from the blood vessel of a subject. Blood may be obtained from the blood vessel by methods well known in the art. Very suitably, the blood samples may be provided by venipuncture using e.g. a vacutainer or by fingersticks sampling. The blood vessel may be a vein or an artery. After removal of the blood sample, the sample is kept for subsequent protein measurement under conditions that avoid RNA or protein breakdown. If required, specific fractions of the blood, such as plasma or serum, and cellular fractions may be separated and analysed individually. Cell fractions may be further subdivided to provide polymorphonuclear leukocytes. The expression products may be detected in any suitable fraction of the blood.

The biomarkers of the present invention may be used in methods of the invention for prognostic diagnosis of cardiovascular events, in particular ischemic cardiovascular events.

Based on the demonstration that specific gene expression profiles and gene products in blood as described above are so closely associated to prognosis of cardiovascular events, the present invention now provides a method for the diagnosis or prognosis of cardiovascular disease in a subject, comprising detecting in the blood of said subject a biomarker according. The presence of said biomarker in said (sample of) blood indicates that the test subject is at risk of an ischemic event.

A method of the invention is preferably performed on a blood sample from a subject (suspected to be) at risk of an ischemic event, although in vivo methods may also be applied. As a reference, a sample from a control subject not at risk of developing an ischemic event may be used. Comparison of these samples may reveal deviant biomarker levels in the test sample. Prior to the availability of the present method, the question whether a subject was at risk of developing an ischemic event, was often revealed after a prolonged period of time. By using the prognostic diagnostic methods of the invention, the results are usually available within a day following the sampling of the blood. But even if the blood reveals the presence of biomarkers as referred to herein that are indicative of (a risk of) ischemia (for instance as predicted from database records), yet, it may take many years before said risk materializes in the form of, for instance, a cardiovascular event.

A method of the invention may include the typing of blood samples according to the risk associated with suffering ischemia. Typing of a blood sample in a method of the invention further comprises the step of measuring the amount of at least one biomarker of the invention in a positive control sample (from a risk patient) and in a negative control sample (from a non-risk patient or historical control or reference) or providing a biomarker profile for both samples. The term “amount of at least one biomarker” as used in this description, may refer to a relative amount or an absolute amount (e.g. a concentration). A positive control sample is also referred to as a reference sample and the amount of the biomarker therein is referred to as the reference value (i.e. above or below which there is a positive identification of the presence of a risk). A negative control sample is also referred to herein as a control sample.

It will be appreciated that the step of measuring the amount of at least one biomarker need not result in an exact determination of the concentration of the RNA of protein representing the biomarker in said sample. It is sufficient that an expression of the amount is obtained relative to the amount present (or not present) in a control sample. Any (semi) quantitative method is suitable, as long as the measured amount can be compared with control or reference values.

In order to identify a candidate biomarker, typing includes the step of determining whether said at least one biomarker is differentially present in a first blood sample compared to a second blood sample, or determining the differential expression profile between a first and a second blood sample. This step may conveniently be performed by using gene expression arrays, or by analysing the RNAs or proteins present in the two blood samples by 2-dimensional poly-acrylamide gel electrophoreses (2-D PAGE) and western blotting or mass spectrometry. Such methods generally involve the partial degradation of the proteins into peptides and the sequencing and subsequent identification of these peptides by tandem-MS. Such methods are well established in the art.

When the differential expression profile is determined, and the amount(s) of biomarker present in the samples that resemble the risk and non-risk condition are determined, the amounts must be correlated to the condition. Statistical analysis thereof involves routine procedures, provided that clinical data for the medical condition under study are properly annotated to the samples analysed.

Finally, the differentially present protein or RNA or differential protein or RNA expression profile is identified as a biomarker when there is indeed a correlation between the occurrence of the medical condition and the presence (or absence) of the biomarker.

The present invention also provides a kit of parts for performing the methods as described above. Such kits of parts are based on the detection of the biomarker by in vivo or ex vivo methods as described above. A kit of parts of the present invention comprises a biomarker, or a detectable binding partner thereof, for instance an antibody that binds specifically to the biomarker.

A kit of parts may further comprise components for validating the detection protocol, such as reference or control samples (activated EPCs and normal circulatory EPCs), information on the reference value (normal healthy value) for the biomarker, peptides capable of binding to the antibody and which can for instance be used in competitive ELISA assays; detectable markers, often containing a labelling moiety, for detecting binding between said biomarker and said antibody.

Labelling moieties may include fluorescent, chemiluminescent, magnetic, radioactive or other moieties suitable for detection by dedicated equipment

The measured concentration may then be compared to reference values available in a database. Such a database may have the form of a listing of expression products, wherein to each expression products is annotated a reference or threshold value below or above which the risk on the occurrence of an ischemic event in a patient is increased. In order to determine the threshold value for each expression product, a comprehensive study may be performed between samples from risk-patients (patients that have suffered an ischemic event either coronary, cerebrovascular or peripheral ischemia) and non-risk patients (that have not suffered an ischemic event), e.g. such as described herein, and wherein the threshold value is the uppermost or lowest value among the non-risk patients, above which, respectively, below which the statistical chance on the occurrence of an ischemic event is significantly increased.

Alternatively, the database may take the form of a collection of one or more reference samples, containing the said at least one biomarker in an amount equal to the reference value for that biomarker. In such instances, the steps of measuring the amount of at least one biomarker in a sample and the step of comparing the measured amount with reference values, may be performed in a single assay wherein the amount of said biomarker in test and control sample is determined relative to each other, for instance by using any available differential expression analysis technique. Any method suitable for analysing the differential expression of proteins between samples may be used in such instance. When the differential expression of a large number of proteins or RNAs is required, antibody or DNA microarrays may suitably be used.

The preparation of an antibody microarray or RNA microchip array on e.g. glass slides is known to the skilled person. Antibodies, respectively probe DNA may be spotted on for instance amino-reactive, respectively silanized glass slides or other functionalized surfaces. Generally, methods are available to the skilled person to print as many as 20000 spots on a single 2.5×7.5 cm glass slide with individual spots being spotted about 300 μm apart. In order to allow the performance of multiple binding experiments on a single slide, a number of grids consisting of a defined group of antibodies, resp. DNA probes, can be spotted on one slide. The antibodies, resp. DNA probes, may be spotted by any available spotting technique, for instance by contact printing. Tools and technologies developed for the production of DNA microarrays, such as spotter, incubation chambers, differential fluorescent labelling techniques and imaging equipment for quantitative measurement of binding studies, are readily available to the artisan. Procedures for the preparation of antibody arrays based on protein or peptide sequences are commercially available, for instance from Eurogentec, Seraing, Belgium.

As stated above, microarrays may be used for differential gene expression studies (protein or RNA profiling). In order to measure the differential expression of expression products in a biological sample under an experimental condition and compare the expression with control samples or reference values, several methods may be used for labelling of the expression products. Very suitable, the expression products from the biological samples are labelled with one or more fluorescent probes (e.g. Cy3 and Cy5) using standard protein or RNA labelling protocols. Once the expression products of a biological sample (test and control) have been labelled (preferably differentially labelled using different colour probes for test and control), they can be brought in contact with the microarray. The binding of the expression products to the antibodies, cDNA or oligonucleotide probes on the array may for instance be performed upon incubation of the microarray slide with a small volume (±50 μl) of labelled biological material, under cover slips. The detection of expression product bound to the microarray may be based on the generation of fluorescence. Expression products that bind to the microarray may then be detected using a fluorescent scanner and individual spots of the microarray can then be analysed to determine the differential expression between the test and control sample.

In alternative procedures, the microarrays may be used as capturing chips for the quantification of multiple expression products in a biological sample using ELISA methods on the chip. The various proteins identified as biomarkers for assessing the risk of an ischemic event as described herein may be measured more quantitatively by such procedures. To determine the concentration of a protein in a biological sample, ELISA techniques are very suitable. Such techniques involve the production of a calibration curve of the fluorescence intensity vs. protein concentration, or the use of a competitive ELISA format, wherein known amounts of unlabelled protein or antigen are provided in the test. When using methods such as peptide immunisation for the preparation of an antibody microarrays as described above, the peptides used for immunisation may be used in competitive ELISA experiments on the microarray. Alternatively, multiple sandwich ELISA can be developed using as second antibody, for instance an antibody raised by peptide immunisation against a second epitope of the target protein (a second synthetic peptide).

In yet another aspect, the present invention provides the use of a biomarker as defined herein above for predicting the risk of an ischemic event in a subject. Such use involves the detection of the biomarker in (a sample of) a patient, and the determination whether the amount detected is above or below the reference value.

### Therapeutic Methods

Biomarkers can be helpful in medical decision making as they can diagnose patients, identify certain risk populations and evaluate the (lack of) proper response to the initiated therapy.

The genes identified herein constitute potentially regulatory genes involved in the regulation of new vessel formation and vessel repair and therefore also constitute a new method to treat cardiovascular disease in general and ischemia (peripheral and myocardial) and atherosclerosis (progression of atherogenesis and stabilization of vulnerable plaques) in particular, as well as to prevent pathological vessel formation (diabetic neoangiogenesis, tumor angiogenesis, atherosclerotic plaque destabilization). In particular, the potential value of these genes in the development (and treatment) of several animal models of atherosclerosis, unstable plaque formation, hind limb ischemia, myocardial ischemia and infarction as well as in tumor angiogenesis is proposed.

Patients may be treated by intervention at the genetic level (interference with RNA, DNA transcription/translation, including but not restricted to siRNA, recombinant viral vectors, transfected cell lines or combinations thereof) or by use of treatment with the protein. It is also proposed herein to use activated EPCs as active therapeutic substance.

Alternatively, the treatment may include interference with the working mechanism/effect of the gene or gene products, for instance by using biological or chemical blockers of receptors that interact with the products of the genes as identified herein or that interfere with the signalling cascade resulting from binding of a biomarker of the present invention and its receptor or ligand.

In yet another aspect, the present invention provides a method of treating a subject (having an increased risk of) suffering from cardiovascular disease, in particular ischemia, atherosclerosis and pathological vessel formation, said method comprising using a biomarker as defined herein above as a therapeutic target or as a therapeutic agent. Preferably, said use of said biomarker as a therapeutic target comprises decreasing the amount of at least one expression product that is over-expressed in subjects (having an increased risk of) suffering from cardiovascular disease, in particular ischemia, atherosclerosis and pathological vessel formation, or increasing the amount of at least one expression product that is under-expressed in subjects (having an increased risk of) suffering from cardiovascular disease, in particular ischemia, atherosclerosis and pathological vessel formation. More preferably the expression of said expression product is stimulated or enhanced, or the function of said expression product is interfered with at the level of the receptor or further downstream in the signalling cascade.

Preferably, said use of said biomarker as a therapeutic agent comprises increasing the amount of at least one expression product that is under-expressed in subjects (having an increased risk of) suffering from cardiovascular disease, in particular ischemia, atherosclerosis and pathological vessel formation, and involves for instance administering said protein to said subject.

Alternatively, the use of said biomarker as a therapeutic agent comprises blocking the receptor-ligand interaction of a signalling cascade wherein said biomarker is either ligand, receptor or a member of the signalling cascade.

The biomarkers as defined herein can be cellular or excreted proteins or nucleic acids. Alternatively the biomarkers as defined herein may take the form of a combined biomarker profile. The ultimate biomarker is the activated EPC as defined herein having the specific gene expression profile with respect to the 26 genes of Table 1. Thus, the activated EPC may also be referred to as a biomarker, and may also be used as a therapeutic agent, for treating cardiovascular diseases.

The present invention also relates to the use of the biomarkers of the present invention as therapeutic targets. Pharmacogenetics and pharmacogenomics aim at determining the genetic determinants linked to diseases. Most of the diseases are multigenic diseases, and the identification of the genes involved therein should allow for the discovery of new targets and the development of new drugs.

Many physiological diseases are targeted by this novel pharmaceutical approach. The risk of suffering from cardiovascular disease, in particular ischemia, atherosclerosis and pathological vessel formation may be viewed as a multigenic disease. The biomarkers of the present invention have been identified as genetic markers for predisposition of the disease. Knowledge of the identity of genes involved in development of cardiovascular disease, in particular ischemia, atherosclerosis and pathological vessel formation ultimately resulting in a fatal ischemic event therefore greatly facilitates the development of prophylactic, therapeutic and diagnostic methods for this disease. Diagnosis of the genes responsible for the risk phenotype in a certain subjects allows for the design of therapies comprising the use of specific drugs, for instance, drugs directed against the proteins encoded by these genes.

It is an aspect of the present invention to use the biomarkers of the present invention and/or the genes encoding these biomarkers for the development of inhibitors directed against the genes and/or their expression products (RNA or protein), in particular in the case of over-expression of the biomarker in the subject at risk or directed against ligands or receptors of the signaling cascades of which the biomarker is a member.

Biomarkers of the present invention may be expressed in a patient to compensate to a failing perfusion (i.e. ischemia). Alternatively, the biomarkers of the invention may in other instances reflect an epiphenomenon of ischemia and their successful downregulation will coincide with successful treatment. In general, the therapeutic application will involve modulation of the gene product.

In one embodiment of this aspect, the inhibitors are antibodies and/or antibody derivatives directed against the expression products of genes encoding the biomarkers. Therapeutic antibodies are for instance useful against gene expression products located on the cellular membrane and can be comprised in a pharmaceutical composition. Also, antibodies may be targeted to intracellular, e.g. cytoplasmic, gene products such as RNA's, polypeptides or enzymes, in order to modulate the activity of these products. Preferably, such antibodies are in the form of intrabodies, produced inside a target cell, preferably a plaque-forming cell including T-cells, endothelial cells, and smooth muscle cells, or cells that are found in atherosclerotic lesions, such as leukocytes, macrophages, foam cells, dendritic cells, and mast cells and T cells. In addition, antibodies may be used for deliverance of at least one toxic compound linked thereto to a target cell.

In a preferred embodiment of the present invention, the inhibitor is a small molecule capable of modulating the activity or interfering with the function of the protein expression product of the genes encoding the biomarkers as defined herein. In addition, small molecules can also be used for deliverance of at least one linked toxic compound to the target cell.

On a different level of inhibition, nucleic acids can be used to block the production of proteins by destroying the mRNA transcribed from respective gene encoding the biomarkers. This can be achieved by antisense drugs, ribozymes or by RNA interference (RNAi). By acting at this early stage in the disease process, these drugs prevent the production of a disease-causing protein. The present invention relates to antisense drugs, such as antisense RNA and antisense oligodeoxynucleotides, ribozymes and RNAi molecules, directed against the genes encoding the boiomarkers.

The expression level of a gene can either be decreased or increased in a risk phenotype. Naturally, inhibitors are used when the expression levels are elevated. However, the present invention also provides for “enhancers”, to boost the expression level of a gene encoding the biomarkers associated with a risk of suffering a cardiovascular event and of which the expression levels are reduced in a risk situation. “Enhancers” may be any chemical or biological compound known or found to increase the expression level of genes, to improve the function of an expression product of a gene or to improve or restore the expression of a gene.

Very suitable therapies to overcome reduced expression levels of a gene or to restore the expression of a gene encoding the biomarkers as disclosed herein include the replacement by gene therapy of the gene or its regulatory sequences that drive the expression of said gene. The invention therefore relates further to gene therapy, in which a dysfunctional gene of a subject encoding the biomarkers or a dysfunctional regulatory sequence of a gene of a subject encoding a biomarker is replaced by a functional counterpart, e.g. by stable integration of for instance a lentiviral vector comprising a functional gene or regulatory sequence into the genome of a subject's host cell which is a progenitor cell of the target cell-line of the subject and grafting of said transfected host cell into said subject.

The invention also relates to forms of gene therapy, in which the genes encoding the biomarker are i.a. used for the design of dominant-negative forms of these genes which inhibit the function of their wild-type counterparts following their directed expression from a suitable vector in a target cell.

Another object of the present invention is to provide a pharmaceutical composition for the treatment of patients having an increased risk of suffering from cardiovascular disease, in particular ischemia, atherosclerosis and pathological vessel formation comprising one or more of the inhibitors, “enhancers”, replacement compounds, vectors or host cells according to the present invention as a pharmaceutical reagent or active ingredient. The composition can further comprise at least one pharmaceutical acceptable additive like for example a carrier, an emulsifier, or a conservative.

In addition, it is the object of the present invention to provide a method for treatment of subjects suffering from an increased risk of suffering cardiovascular disease, in particular ischemia, atherosclerosis and pathological vessel formation which method comprises the administration of the pharmaceutical composition according to the invention to patients in need thereof in a therapeutically effective amount.

### Small Molecule Inhibitors

Small molecule inhibitors are usually chemical entities that can be obtained by screening of already existing libraries of compounds or by designing compounds based on the structure of the protein encoded by a gene involved in tumor development. Briefly, the structure of at least a fragment of the protein is determined by either Nuclear Magnetic Resonance or X-ray crystallography. Based on this structure, a virtual screening of compounds is performed. The selected compounds are synthesized using medicinal and/or combinatorial chemistry and thereafter analyzed for their inhibitory effect on the protein in vitro and in vivo. This step can be repeated until a compound is selected with the desired inhibitory effect. After optimization of the compound, its toxicity profile and efficacy as therapeutic is tested in vivo using appropriate animal model systems.

Differentially expressed genes that do not encode membrane-bound proteins are selected as targets for the development of small molecule inhibitors. To identify putative binding sites or pockets for small molecules on the surface of the target proteins, the three-dimensional structure of those targets are determined by standard crystallization techniques. Additional mutational analysis may be performed to confirm the functional importance of the identified binding sites. Subsequently, Cerius2 (Molecular Simulations Inc., San Diego, Calif., USA) and Ludi/ACD (Accelrys Inc., San Diego, Calif., USA) software is used for virtual screening of small molecule libraries. The compounds identified as potential binders by these programs are synthesized by combinatorial chemistry and screened for binding affinity to the targets as well as for their inhibitory capacities of the target protein's function by standard in vitro and in vivo assays. In addition to the rational development of novel small molecules, existing small molecule compound libraries are screened using these assays to generate lead compounds. Lead compounds identified are subsequently co-crystallized with the target to obtain information on how the binding of the small molecule can be improved. Based on these findings, novel compounds are designed, synthesized, tested, and co-crystallized. This optimization process is repeated for several rounds leading to the development of a high-affinity compound of the invention that successfully inhibits the function of its target protein. Finally, the toxicity of the compound is tested using standard assays (commercially available service via MDS Pharma Services, Montreal, Quebec, Canada) after which it is screened in an animal model system.

### Ribozymes

Trans-cleaving catalytic RNAs (ribozymes) are RNA molecules possessing endoribonuclease activity. Ribozymes are specifically designed for a particular target, and the target message must contain a specific nucleotide sequence. They are engineered to cleave any RNA species site-specifically in the background of cellular RNA. The cleavage event renders the mRNA unstable and prevents protein expression. Importantly, ribozymes can be used to inhibit expression of a gene of unknown function for the purpose of determining its function in an in vitro or in vivo context, by detecting the phenotypic effect.

One commonly used ribozyme motif is the hammerhead, for which the substrate sequence requirements are minimal. Design of the hammerhead ribozyme is well known in the art, as is the therapeutic uses of ribozymes. Ribozymes can for instance be prepared and used as described in U.S. Pat. No. 5,254,678. Ribozyme cleavage of HIV-I RNA is described in U.S. Pat. No. 5,144,019; methods of cleaving RNA using ribozymes is described in U.S. Pat. No. 5,116,742; and methods for increasing the specificity of ribozymes are described in U.S. Pat. No. 5,225,337. Preparation and use of ribozyme fragments in a hammerhead or hairpin structure is also known in the art. Ribozymes can also be made by rolling transcription.

The hybridizing region of the ribozyme may be modified or may be prepared as a branched structure. The basic structure of the ribozymes may also be chemically altered in ways familiar to those skilled in the art, and chemically synthesized ribozymes can be administered as synthetic oligonucleotide derivatives modified by monomeric units. In a therapeutic context, liposome mediated delivery of ribozymes improves cellular uptake.

Therapeutic and functional genomic applications of ribozymes proceed beginning with knowledge of a portion of the coding sequence of the gene to be inhibited. Thus, for many genes, a nucleic acid sequence provides adequate sequence for constructing an effective ribozyme. A target cleavage site is selected in the target sequence, and a ribozyme is constructed based on the 5′ and 3′ nucleotide sequences that flank the cleavage site. Retroviral vectors are engineered to express monomeric and multimeric hammerhead ribozymes targeting the mRNA of the target coding sequence. These monomeric and multimeric ribozymes are tested in vitro for an ability to cleave the target mRNA. A cell line is stably transduced with the retroviral vectors expressing the ribozymes, and the transduction is confirmed by Northern blot analysis and reverse-transcription polymerase chain reaction (RT-PCR). The cells are screened for inactivation of the target mRNA by such indicators as reduction of expression of disease markers or reduction of the gene product of the target mRNA.

### Antisense

Antisense polynucleotides are designed to specifically bind to RNA, resulting in the formation of RNA-DNA or RNA-RNA hybrids, with an arrest of DNA replication, reverse transcription or messenger RNA translation. Antisense polynucleotides based on a selected sequence can interfere with expression of the corresponding gene.

Antisense polynucleotides are typically generated within the cell by expression from antisense constructs that contain the antisense strand as the transcribed strand. Antisense polynucleotides will bind and/or interfere with the translation of the corresponding mRNA. As such, antisense may be used therapeutically the inhibit the expression of oncogenes.

Antisense RNA or antisense oligodeoxynucleotides (antisense ODNs) can both be used and may also be prepared in vitro synthetically or by means of recombinant DNA techniques. Both methods are well within the reach of the person skilled in the art. ODNs are smaller than complete antisense RNAs and have therefore the advantage that they can more easily enter the target cell. In order to avoid their digestion by DNAse, ODNs and antisense RNAs may be chemically modified. For targeting to the desired target cells, the molecules may be linked to ligands of receptors found on the target cells or to antibodies directed against molecules on the surface of the target cells.

### RNAi

RNAi refers to the introduction of homologous double stranded RNA to specifically target the transcription product of a gene, resulting in a null or hypomorphic phenotype. RNA interference requires an initiation step and an effector step. In the first step, input double-stranded (ds) RNA is processed into nucleotide ‘guide sequences’. These may be single- or double-stranded. The guide RNAs are incorporated into a nuclease complex, called the RNA-induced silencing complex (RISC), which acts in the second effector step to destroy mRNAs that are recognized by the guide RNAs through base-pairing interactions. RNAi molecules are thus double stranded RNAs (dsRNAs) that are very potent in silencing the expression of the target gene. The invention provides dsRNAs complementary to the genes encoding the biomarkers of the present invention.

The ability of dsRNA to suppress the expression of a gene corresponding to its own sequence is also called post-transcriptional gene silencing or PTGS. The only RNA molecules normally found in the cytoplasm of a cell are molecules of single-stranded mRNA. If the cell finds molecules of double-stranded RNA, dsRNA, it uses an enzyme to cut them into fragments containing in general 21-base pairs (about 2 turns of a double helix). The two strands of each fragment then separate enough to expose the antisense strand so that it can bind to the complementary sense sequence on a molecule of mRNA. This triggers cutting the mRNA in that region thus destroying its ability to be translated into a polypeptide. Introducing dsRNA corresponding to a particular gene will knock out the cell's endogenous expression of that gene. This can be done in particular tissues at a chosen time. A possible disadvantage of simply introducing dsRNA fragments into a cell is that gene expression is only temporarily reduced. However, a more permanent solution is provided by introducing into the cells a DNA vector that can continuously synthesize a dsRNA corresponding to the gene to be suppressed.

RNAi molecules are prepared by methods well known to the person skilled in the art. In general an isolated nucleic acid sequence comprising a nucleotide sequence which is substantially homologous to the sequence of at least one of the genes encoding the biomarkers of the invention and which is capable of forming one or more transcripts able to form a partially of fully double stranded (ds) RNA with (part of) the transcription product of said genes will function as an RNAi molecule. The double stranded region may be in the order of between 10-250, preferably 10-100, more preferably 20-50 nucleotides in length.

RNAi molecules are preferably expressed from recombinant vectors in transduced host cells, hematopoietic stem cells being very suitable thereto.

### Dominant Negative Mutations

Dominant negative mutations are readily generated for corresponding proteins that are active as multimers. A mutant polypeptide will interact with wild-type polypeptides (made from the other allele) and form a non-functional multimer. Thus, a mutation is in a substrate-binding domain, a catalytic domain, or a cellular localization domain. Preferably, the mutant polypeptide will be overproduced. Point mutations are made that have such an effect. In addition, fusion of different polypeptides of various lengths to the terminus of a protein can yield dominant negative mutants. General strategies are available for making dominant negative mutants. Such a technique can be used for creating a loss of function mutation, which is useful for determining the function of a protein.

### Use of Polypeptides to Raise Antibodies

The present invention provides in one aspect for antibodies suitable for therapeutic and/or diagnostic use.

Therapeutic antibodies include antibodies that can bind specifically to the expression products of the genes encoding the biomarkers of the invention. By binding directly to the gene products, the antibodies may influence the function of their targets by, for example, in the case of proteins, steric hindrance, or by blocking at least one of the functional domains of those proteins. As such, these antibodies may be used as inhibitors of the function of the gene product. Such antibodies may for instance be generated against functionally relevant domains of the proteins and subsequently screened for their ability to interfere with the target's function using standard techniques and assays.

Alternatively, anti-RNA antibodies may for instance be useful in silencing messengers of the tumor-related genes of the present invention. In another alternative, antibodies may also be used to influence the function of their targets indirectly, for instance by binding to members of signaling pathways in order to influence the function of the targeted proteins or nucleic acids. In yet another alternative, therapeutic antibodies may carry one or more toxic compounds that exert their effect on the target or target cell by virtue of the binding of the carrying antibody thereto.

For diagnostic purposes, antibodies similar to those above, preferably those that are capable of binding to the expression products of the genes of the present invention may be used, and that are provided with detectable labels such as fluorescent, luminescent, or radio-isotope labels in order to allow the detection of the gene product. Preferably such diagnostic antibodies are targeted to proteinaceous targets present on the outer envelop of the cell, such as membrane bound target proteins (biomarkers).

The antibodies used in the present invention may be from any animal origin including birds and mammals (e.g., human, murine, donkey, sheep, rabbit, goat, guinea pig, camel, horse, or chicken). Preferably, the antibodies of the invention are human or humanized monoclonal antibodies. As used herein, “human” antibodies include antibodies having the amino acid sequence of a human immunoglobulin and include antibodies isolated from human immunoglobulin libraries (including, but not limited to, synthetic libraries of immunoglobulin sequences homologous to human immunoglobulin sequences) or from mice that express antibodies from human genes.

For some uses, including in vivo therapeutic or diagnostic use of antibodies in humans and in vitro detection assays, it may be preferred to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences or synthetic sequences homologous to human immunoglobulin sequences. See also U.S. Pat. Nos. 4,444,887 and 4,716,111; and PCT publications WO 98/46645, WO 98/50433, WO 98/24893 and WO98/16654, each of which is incorporated herein by reference in its entirety.

The antibodies to be used with the methods of the invention include derivatives that are modified, i.e, by the covalent attachment of any type of molecule to the antibody such that covalent attachment. Additionally, the derivative may contain one or more non-classical amino acids.

In certain embodiments of the invention, the antibodies to be used with the invention have extended half-lives in a mammal, preferably a human, when compared to unmodified antibodies. Antibodies or antigen-binding fragments thereof having increased in vivo half-lives can be generated by techniques known to those of skill in the art (see, e.g., PCT Publication No. WO 97/34631).

In certain embodiments, antibodies to be used with the methods of the invention are single-chain antibodies. The design and construction of a single-chain antibody is well known in the art.

In certain embodiments, the antibodies to be used with the invention bind to an intracellular epitope, i.e., are intrabodies. An intrabody comprises at least a portion of an antibody that is capable of immunospecifically binding an antigen and preferably does not contain sequences coding for its secretion. Such antibodies will bind its antigen intracellularly. In one embodiment, the intrabody comprises a single-chain Fv (“sFv”). In a further embodiment, the intrabody preferably does not encode an operable secretory sequence and thus remains within the cell.

Generation of intrabodies is well-known to the skilled artisan and is described for example in U.S. Pat. Nos. 6,004,940; 6,072,036; 5,965,371, which are incorporated by reference in their entireties herein.

In one embodiment, intrabodies are expressed in the cytoplasm. In other embodiments, the intrabodies are localized to various intracellular locations. In such embodiments, specific localization sequences can be attached to the intranucleotide polypepetide to direct the intrabody to a specific location.

The antibodies to be used with the methods of the invention or fragments thereof can be produced by any method known in the art for the synthesis of antibodies, in particular, by chemical synthesis or preferably, by recombinant expression techniques.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art. The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Examples of phage display methods that can be used to make the antibodies of the present invention include those disclosed in WO97/13844; and U.S. Pat. Nos. 5,580,717, 5,821,047, 5,571,698, 5,780,225, and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324.

It is also possible to produce therapeutically useful IgG, IgA, IgM and IgE antibodies. For a detailed discussion of the technology for producing human antibodies and human monoclonal antibodies and protocols for producing such antibodies, see, e.g., PCT publication No. WO 98/24893, which is incorporated by reference herein in its entirety. In addition, companies such as Medarex, Inc. (Princeton, N.J.), Abgenix, Inc. (Freemont, Calif.) and Genpharm (San Jose, Calif.) can be engaged to provide human antibodies directed against a selected antigen using technology similar to that described above.

Recombinant expression used to produce the antibodies, derivatives or analogs thereof (e.g., a heavy or light chain of an antibody of the invention or a portion thereof or a single chain antibody of the invention), requires construction of an expression vector containing a polynucleotide that encodes the antibody and the expression of said vector in a suitable host cell or even in vivo. Once a polynucleotide encoding an antibody molecule or a heavy or light chain of an antibody, or portion thereof (preferably, but not necessarily, containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules as defined herein

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc.

Once an antibody molecule to be used with the methods of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

As stated above, according to a further aspect, the invention provides an antibody as defined above for use in therapy.

For therapeutic treatment, antibodies may be produced in vitro and applied to the subject in need thereof. The antibodies may be administered to a subject by any suitable route, preferably in the form of a pharmaceutical composition adapted to such a route and in a dosage which is effective for the intended treatment. Therapeutically effective dosages of the antibodies required for decreasing the rate of progress of the disease or for eliminating the disease condition can easily be determined by the skilled person.

Alternatively, antibodies may be produced by the subject itself by using in vivo antibody production methodologies as described above. Suitably, the vector used for such in vivo production is a viral vector, preferably a viral vector with a target cell selectivity for specific target cell referred to herein.

Therefore, according to a still further aspect, the invention provides the use of an antibody as defined above in the manufacture of a medicament for use in the treatment of a subject to achieve the said therapeutic effect. The treatment comprises the administration of the medicament in a dose sufficient to achieve the desired therapeutic effect. The treatment may comprise the repeated administration of the antibody.

According to a still further aspect, the invention provides a method of treatment of a human comprising the administration of an antibody as defined above in a dose sufficient to achieve the desired therapeutic effect. The therapeutic effect being the alleviation or prevention of the risk of suffering a cardiovascular event.

The diagnostic and therapeutic antibodies are preferably used in their respective application for the targeting of kinases or phosphatases, which are often coupled to receptor molecules on the cell's surface. As such, antibodies capable of binding to these receptor molecules can exert their activity-modulating effect on the kinases or phosphatases by binding to the respective receptors. Also transporter proteins may be targeted with advantage for the same reason that the antibodies will be able to exert their activity-modulating effect when present extracellularly. The above targets, together with signaling molecules, represent preferred targets for the antibody uses of the invention as more effective therapy and easier diagnosis is possibly thereby.

The diagnostic antibodies can suitably be used for the qualitative and quantitative detection of gene products, preferably proteins in assays for the determination of altered levels of proteins or structural changes therein. Protein levels may for instance be determined in cells, in cell extracts, in supernatants, body fluids by for instance flow-cytometric evaluation of immunostained target cells, preferably in blood or in endothelial progenitor cells (EPCs) or polymorphonuclear leukocytes (PMNs) present in said blood. Alternatively, quantitative protein assays such as ELISA or RIA, Western blotting, and imaging technology (e.g., using confocal laser scanning microscopy) may be used in concert with the antibodies as described herein for the diagnosis of an increased risk on cardiovascular events.

### Pharmaceutical Compositions and Therapeutic Uses

Pharmaceutical compositions can comprise polypeptides, antibodies, polynucleotides (antisense, RNAi, ribozyme), or small molecules of the claimed invention, collectively called inhibitor compounds herein. The pharmaceutical compositions will comprise a therapeutically effective amount of either a biomarker protein, an antibody, a polynucleotides or small molecules as described herein.

Inhibitor compounds may also include substances capable of (chemical) interference with the function of the identified regulatory genes, for instance through receptor blockage. Alternatively one may employ decoy technology for transcription factors such as for instance described in U.S. Pat. No. 6,774,118, which is referred to herein by reference in its entirety.

An inhibitor of a biomarker may be an antibody against the biomarker, antobodies against a receptor of said biomarker, biomarker-bnding proeins, or isoforms muteins, fused proteins, or functional derivatives thereof inhibiting the biological activity of the biomarker.

The skilled person may also find genes that are down-regulated in an activated EPC as defined herein and may find suitable use of these down-regulated genes or their expression products as therapeutic targets or therapeutic agents in aspects of the present invention. Inhibitors of expression products that are downregulated in activated cells, may suitably be used as therapeutic agents in methods for the treatment of cardiovascular disease.

The term “therapeutically effective amount” as used herein refers to an amount of a therapeutic agent to treat, ameliorate, or prevent a desired disease or condition, or to exhibit a detectable therapeutic or preventative effect. The effect can be detected by, for example, chemical markers or antigen levels. Therapeutic effects also include reduction in physical symptoms, such as decreased body temperature. The precise effective amount for a subject will depend upon the subject's size and health, the nature and extent of the condition, and the therapeutics or combination of therapeutics selected for administration. Thus, it is not useful to specify an exact effective amount in advance. However, the effective amount for a given situation can be determined by routine experimentation and is within the judgment of the clinician. Specifically, the compositions of the present invention can be used to treat, ameliorate, or prevent the occurrence of a cardiovascular event in a subject and/or accompanying biological or physical manifestations.

For purposes of the present invention, an effective dose will be from about 0.01 mg/kg to 50 mg/kg or 0.05 mg/kg to about 10 mg/kg of the polynucleotide, polypeptide or antibody compositions in the individual to which it is administered.

A pharmaceutical composition can also contain a pharmaceutically acceptable carrier. The term “pharmaceutically acceptable carrier” refers to a carrier for administration of a therapeutic agent, such as antibodies or a polypeptide, genes, and other therapeutic agents. The term refers to any pharmaceutical carrier that does not itself induce the production of antibodies harmful to the individual receiving the composition, and which may be administered without undue toxicity. Suitable carriers may be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art.

Pharmaceutically acceptable salts can be used therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients is available in Remington's Pharmaceutical Sciences (Mack Pub. Co., N.J. 1991).

Pharmaceutically acceptable carriers in therapeutic compositions may contain liquids such as water, saline, glycerol and ethanol. Additionally, auxiliary substances, such as wetting or emulsifying agents, pH buffering substances, and the like, may be present in such vehicles. Typically, the therapeutic compositions are prepared as injectables, either as liquid solutions or suspensions; solid forms suitable for solution in, or suspension in, liquid vehicles prior to injection may also be prepared. Liposomes are included within the definition of a pharmaceutically acceptable carrier.

### Delivery Methods

Once formulated, the pharmaceutical compositions of the invention can be (1) administered directly to the subject; (2) delivered ex vivo, to cells derived from the subject; or (3) delivered in vitro for expression of recombinant proteins.

Direct delivery of the compositions will generally be accomplished by injection, either subcutaneously, intraperitoneally, intravenously or intramuscularly, or delivered to the interstitial space of a tissue. The compositions can also be administered into a plaque or lesion. Other modes of administration include topical, oral, catheterized and pulmonary administration, suppositories, and transdermal applications, needles, and particle guns or hyposprays. Dosage treatment may be a single dose schedule or a multiple dose schedule.

Methods for the ex vivo delivery and reimplantation of transformed cells into a subject are known in the art and described in e.g., International Publication No. WO 93/14778. Examples of cells useful in ex vivo applications include, for example, stem cells, particularly hematopoetic, lymph cells, macrophages, dendritic cells, or tumor cells.

Generally, delivery of nucleic acids for both ex vivo and in vitro applications can be accomplished by, for example, dextran-mediated transfection, calcium phosphate precipitation, Polybrene® mediated transfection, protoplast fusion, electroporation, encapsulation of the polynucleotide(s) in liposomes, and direct microinjection of the DNA into nuclei, all well known in the art.

Various methods are used to administer the therapeutic composition directly to a specific site in the body. For example, a target location is located and the therapeutic composition injected in the target directly. Alternatively, arteries which serve target location are identified, and the therapeutic composition injected into such an artery, in order to deliver the composition directly into the target location. The antisense composition is directly administered to the surface of an atherosclerotic lesion, for example, by topical application of the composition. X-ray imaging is used to assist in certain of the above delivery methods.

Receptor-mediated targeted delivery of therapeutic compositions containing an antisense polynucleotide, subgenomic polynucleotides, or antibodies to specific tissues is also used. Receptor-mediated DNA delivery techniques are well known in the art. Preferably, receptor-mediated targeted delivery of therapeutic compositions containing antibodies of the invention is used to deliver the antibodies to specific tissue.

Pharmaceutical compositions containing antisense, ribozyme or RNAi polynucleotides are administered in a range of about 100 ng to about 200 mg of polynucleotides for local administration in a gene therapy protocol. Concentration ranges of about 500 ng to about 50 mg, about 1 μg to about 2 mg, about 5 μg to about 500 μg, and about 20 μg to about 100 μg of polynucleotides can also be used during a gene therapy protocol. Factors such as method of action and efficacy of transformation and expression are considerations which will affect the dosage required for ultimate efficacy of the polynucleotides. Where greater expression is desired over a larger area of tissue, larger amounts of polynucleotides or the same amounts readministered in a successive protocol of administrations, or several administrations to different adjacent or close tissue portions of, for example, an atherosclerotic site, may be required to effect a positive therapeutic outcome. In all cases, routine experimentation in clinical trials will determine specific ranges for optimal therapeutic effect. A more complete description of gene therapy vectors, especially retroviral vectors, is contained in U.S. Ser. No. 08/869,309, which is expressly incorporated herein.

All reference referred to herein are incorporated by reference herein in their entirety.

The present invention will now be further illustrated in the Experimental part described below.

### EXPERIMENTAL PART

### Example 1

Revascularization through angiogenesis may constitute an attractive treatment strategy for critical limb ischemia and ischemic heart disease. We have identified new molecular pathways through which to control vasculogenesis and tested their ability to restore vascular function in appropriate animal models. Through a DNAchip microarray analysis, we identified 1160 differentially expressed clones, associated with the different phases of vasculogenesis in mouse development. We then conbined the complementary strength of mouse and zebrafish genomic studies to identify among those genes, key selector genes for vasculogenesis. The genes obtained from microarrays were used (1) to obtain their zebrafish orthologues and to perform whole mount in situ hybridizations in fish embryos to identify their expression patterns, and (2) to use antisense morpholinos in zebrafish to knock down those genes that are specifically expressed in angioblasts and vessels. Roughly 30 genes have passed these filters (1) and (2). We further studied the effects of these genes of interest by ectopic expression and knock down analysis, in vitro in 3D matrigel endothelial cell culture, and in vivo, in zebrafish development and in mice models of limb ischemia, atherosclerosis, vulnerable plaque formation, acute myocartdial infarction and tumor angiogenesis. Functional implications in these mice are being studied including quantitative histology analysis, laser doppler imaging, and angiography. Complementary quantitative PCR analysis of RNA expression isolated from circulating PML from blood samples verified expression of the identified candidate vasculogenesis genes in patients with ischemic cardiovascular disease. Several candidate genes with a putative regulatory role have already been identified, including the heme oxygenase family (Hmox1/2), stab1 and 2.

Since the Hmox system was identified as one of the upregulated gene systems during embryonic vasculogenesis, we are studying the role of the heme oxygenase system in vasculogenesis using our generated Hmox1-knockout mice. We postulate that these candidate genes and gene products provide an compensatory system during ischemia and arterial repair leading to compensatory vasculogenesis and vascular repair.

Based on these studies of embryology and ischemia in the zebrafish, mice and cardiovascular patients, we have gained an imperative insight in the molecular mechanisms of vasculogenesis and angioblast differentiation in ischemic disease and arterial repair and identified these genes as indicators of ongoing vasculogenesis and arterial repair following ischemia and arterial injury in a broad setting.

Based on studies of embryology and ischemia, we can identify regulatory genes and signaling pathways involved in the initiation of vasculogenesis, maturation and remodeling of the neocapillary network into a hemodynamic significant arterial bed. These regulatory genes are involved in EPC recruitment, activation and migration into areas of neovascularization due to ischemia and arterial injury. During early embryogenesis, vasculogenesis can be studied independent from intra-embryonic haematopoiesis, during a period in which the initial basic vascular pattern is established, prior to intra-embryonic haematopoiesis. Known and unknown genes (EST tags) involved in these different stages of mouse vasculogenesis were identified by genome-wide screening using DNA-microarrays and have been further selected using expression profiling by quantitative PCR in angioblasts during development and, ischemia in mouse and CAD patients. These candidate genes comprise transcription factors, growth factors and protein kinases and phosphatises. We have combined the complementary strength of mouse and zebrafish genomic studies to identify among those genes, key selector genes for vasculogenesis and thus also involved in arterial repair and ischemia-driven EPC activation and subsequent neovascularization. Subsequently, the genes identified by microarrays have been used to perform whole mount in situ hybridizations in fish embryos to identify their expression patterns during development, and to use antisense morpholinos in zebrafish to knock down those genes that are specifically expressed in angioblasts and vessels. 64 Genes have by this approach passed these selection criteria (i.e. specific angioblasts expression and vasculogenesis phenotype upon silencing).

The role of these 64 candidate genes in vasculogenesis and arterial repair was further explored and verified in vitro in a 3D matrigel EC system using gene transfer analysis by viral vector mediated overexpression and silencing of the target genes. In vivo implications of these vasculogenic factors in the mammalian system were tested by viral vector-mediated gene transfer of candidate genes in a standardized mouse limb ischemia model, atherosclerosis model, a standardized model of vulnerable plaque formation and in a tumor angiogenesis model. The effect of sustained expression or silencing of these novel angiotrophic and maturation factors on neovascularization are monitored by (confocal) histological analysis, laser doppler imaging and angiography.

The skilled person will understand how to further investigate and perform clinical validation studies of the aspects herein proposed by expression of these vasculogenesis genes in a mouse model of acute myocardial infarction and by expression analysis of gene products in various subgroups of cardiovascular patients including, but not limited to stable angina pectoris, unstable angina pectoris, acute coronary syndrome, patients undergoing transient ischemic cerebrovascular events (TIA/CVA), peripheral vascular disease, and patients with refractory angina pectoris.

Currently, the present inventors have identified 64 clones which are expressed specifically during development in murine and piscine angioblasts and during adult ischemia, including the genes encoding Hmox1, and stab 1 and 2.

As Hmox1 was identified as a possible key regulatory protein expressed during murine and piscine embryonic vasculogenesis, the role of the Hmox system was further studied in depth by analysis of the in vitro in vascular plexus formation in Hmox1^{-/-} embryoid bodies. In vivo implications of loss of Hmox1 expression was subsequently tested in the ischemic leg model in Hmox1-nullizygous mice and by use of siRNA knockdown analysis and compared to wild type littermates or scrambled siRNA treated animals.

In order to identify genetic determinants of vasculogenesis, we have performed a genome-wide screen for candidate regulatory genes using DNA chip analysis of the various stages of vascular development during mouse embryogenesis. To identify angioblasts committed to the endothelial cell lineage, we used Flk1-aided cell sorting, which previously have been shown to designate both early haematopoietic stem cells, dedicated angioblasts, as well as fully differentiated endothelial cells, thereby encompassing the full differentiation process from haemangioblast to EC. Immunolabeling studies at different embryonic days indicated that Flk1 was expressed as early as 8 days post-conception (dpc) and cross-correlated with PECAM, vWF, E-selectin, Tie-2, and VE-cadherin expression during later phases of embryogenesis. Likewise, Drake and coworkers demonstrated that morphogenesis of blood vessels could be defined in terms of a sequential expression pattern in which TAL1 and Flk1 are expressed first, followed by PECAM, CD34, VE-cadherin, and later Tie2, suggesting that Flk1+ cells are indeed committed to the endothelial lineage. In contrast, Flk1 expression is rapidly down-regulated in extra-embryonic haematopoietic stem cells as they commit to the haematopoietic lineage.

In situ hybridization and immunolocalization studies indicated that Flk1 transcript is first detectable in the mouse embryo early, on day 8 pc, just before the onset of somitogenesis. At this stage, Flk1/CD34 transcripts are detectable in presumptive vascular endothelial progenitor cells before the formation of definitive blood vessels. Later, Flk1 transcripts are detected in pre-endothelial cells of the developing dorsal aortae and in cranial pre-endothelial cells that later coalesce to form a network of vessels. Early on day 9 of development, the cranial mesenchyme contained many stained pre-endothelial cells forming a capillary network. More important, during these early stages of development, vasculogenesis can be observed in the absence of haematopoiesis, since in the developing mouse intra-embryonic vasculogenesis precedes haematopoiesis, which is observed from 11.5 dpc onwards. Therefore, this time window (day 8-11 dpc) provides a unique opportunity to study vasculogenesis in vivo in the setting of normal mouse development and in the absence of haematopoiesis.

In this study of early mouse embryogenesis, we have isolated Flk1+ angioblasts and Flk1− control cells by flow cytometry cell sorting from FVB/n mouse embryos at days 8, 9, 10, 11 and 16 dpc. Total RNA was isolated and screened for differential RNA expression using DNAchip arrays. In the first selection, we have included genes upregulated two-fold and up. Only genes with a reliable hybridization pattern (based on match-mismatch profile) were selected for further analysis (Rosetta Resolver). Unknown genes (EST tags without defined function or expression profile) were included in the analysis, whereas known structural and household genes were excluded from further analysis. The full open reading frame of selected EST clones were sequenced and further analyzed using homology searches, cluster and domain analyses (CELERA). Since we are predominantly interested in regulatory proteins (and signaling pathways), in particular protein kinases and phosphatases were selected. A total of 1160 known and unknown clones were selected for further in vivo analysis in the zebrafish development based on sequence analysis, literature searches for related functions and preliminary expression data in animal models. A similar transspecies expression profiling approach was previously shown to be successful in the analysis of the genetic regulation of human embryonic haematopoiesis which resulted in 23% of the human clones in a clear phenotype in zebrafish embryos using morpholinos injections, demonstrating the efficiency and feasibility of this combined human/piscine expression profiling approach.

In order to further select the candidate regulatory genes and to demonstrate relevancy of the selected candidate genes in adult vasculogenesis and human disease, the embryonic expression data were cross correlated with the endogenous gene expression in zebrafish development, in an adult mouse model of hind limb ischemia, and in human disease using quantitative (RT)PCR (QPCR).

To further select candidate regulatory genes, expression of candidate genes in the developing vascular tree were verified during zebrafish embryogenesis. We have identified 1160 zebrafish orthologues of those selected genes and have converted them into antisense riboprobes (obtained from IMAGE database, others were cloned by RT PCR). Whole mount in situ hybridization (WISH) have been carried out on various stages of zebrafish angioblast migration and vasculogenesis. Zebrafish are particularly suited for this approach, as there is no need to section the material and as different stages of vessel formation can be combined and analysed in one WISH. These studies have narrowed the selection to 73 clones out of 1160 candidate genes based on their expression pattern in zebrafish embryogenesis in the developing vascular tree and relevant disease models.

In order to demonstrate relevancy of the selected candidate genes in adult vasculogenesis, we have subsequently verified endogenous expression of selected clones by QPCR in an in vivo mouse model of limb ischemia (ligated femoral artery) according to the method described by Couffinhal and co-workers (Circulation [1999] Vol. 99(24):3188-98; Am J Pathol [1998] Vol. 152(6):1667-79).

To demonstrate relevancy of the selected candidate genes in human cardiovascular disease, we are currently verifying expression in isolated human circulating endothelial progenitor cells (EPC) collected from patients admitted with an acute coronary syndrome by QPCR and microarray analysis. Circulating EPC have been shown to be involved in the adult vasculogenesis response and arterial repair in patients with cardiovascular disease and are substantially elevated 2-7 days following acute coronary syndrome suggestive of ongoing ischemic vasculogenesis. EPC analysis and isolation are routinely performed by our laboratory by flow cytometric cell sorting. In addition, differential gene expression of selected clones is also verified in patient material acquired from heart explants by QPCR (CAD). These human samples are routinely collected and stored, and are therefore readily available.

This sub study have narrowed the number of selected vasculogenic clones to 64 candidate genes and confirmed that these clones are equally affected in other models of adult vasculogenesis and in human CAD disease.

Genes differentially expressed during embryonic and, ischemic and tumor vasculogenesis or PEC have been further assessed in vivo in zebrafish development by a morpholino-based knock down approach. 64 candidate genes derived from selection filter 1 (expression analysis in zebrafish development, mouse ischemia model and human CAD disease) are being subjected to functional analysis using a reverse genetics, antisense approach. Zebrafish provide an excellent model to study the genetics of vasculogenesis, since zebrafish embryos develop outside the uterus and undergo vasculogenesis within 24 hours, whereas the genes controlling this process seem to be conserved across species boundaries. In addition, in lower vertebrates, including fish, the use of a particular antisense chemistry (called morpholinos) has proven to provide an easy and robust way to silence specific gene expression by either inhibition of translation or interferinge with splicing of the targeted mRNA, depending on whether the morpholino is directed against the translation start codon or an exon-intron boundary, respectively. Morpholinos against all zebrafish genes have been designed that, as outlined, are expressed in angioblasts and vessels during development, and are used to study the effect in embryos transgenic for fli::GFP and GATA2::GFP. These fli::GFP transgenic fish express the fluorescent GFP protein in the endothelial cells which allows visualization and analysis of vessel formation in the life embryo at various stages. This research strategy was taken to analyse for genes with a suspected role in human, mouse and piscine vasculogenic stem cells differentiation, and led to the identification of the 64 out of 73 genes that were initially identified through a transcriptional profiling approach in humans which show a dramatic phenotype in zebrafish embryos when knocked down.

Knockdown analysis of potentially regulatory genes of vasculogenesis resulted in various interesting vasculogenic phenotypes, including, loss of vasculogenesis (a-vasculogenesis), exuberant vasculogenesis or aberrant vessel morphology with loss of integrity, as demonstrated by GFP expression, abnormal circulation and vascular integrity in the Fli::GFP zebrafish.

For instance, morpholino-induced knockdown analysis of Sox7/18, two candidate genes identified by DNAarray and ISH analysis, resulted in a phenotype with prominent shunt formation between dorsal aorta and great cardiac vein (suggesting aberrant tubulogenesis), edema formation and deficient circulation in Fli::GFP zebrafish.

### Example 2

### Genes Differentially Expressed During Embryonic and Ischemic Vasculogenesis: In Vitro Studies in the 3D Matrigel Array; In Vivo Studies in a Mammalian Hind Limb Ischemia Model and a Mouse Model of Atherosclerosis (and Plaque Destabilization), Tumor Angiogenesis and Acute Myocardial Infarction

To further select and define the specific role of candidate genes during mammalian vasculogenesis, selected genes involved in the regulation of vasculogenesis, as identified with previous DNA-microarray analysis, QPCR studies, and WISH analysis in zebrafish, and morpholino knock down analysis in zebra fish development, can be analyzed using gain-of-function and loss-of-function modifications in an in vitro vasculogenesis model (using recombinant viral vector-mediated gene transfer and gene silencing by siRNA). As an in vitro vasculogenesis model, we use the 3D matrigel system. The differentiation of genetically modified EC and EPC cells, in which gain-of-function or loss-of-function modifications have been introduced, offer excellent alternatives to in vivo studies on transgenic animals to analyze the consequences of specific mutations on the process of vascular development, especially when these mutations are lethal to embryos. A lenti- and adenoviral vector system (feline immunodeficiency vector system—FIV, Ad5) are used to express genes of interest in cell cultures, and animals models, as somatic transgenic model, which allows the study of the in vivo function of genes of interest in the pathogenesis of ischemic disease.

Genes involved in the regulation of vasculogenesis, as identified with DNA-microarray analysis, QPCR studies, and WISH analysis in zebrafish, are overexpressed in murine and human endothelial cells and endothelial progenitor cells using a recombinant viral vector system. Vascular morphometry is analyzed using confocal microscopy combined with computer-assisted image analysis (using 2D rendering). Cell cycle progression, cell apoptosis, and metalloprotease protein content were analyzed using cell flow cytometry using Flk1/propidium iodine, Flk1/Annexin-V-fluos and Flk1/MMP9 double labeling respectively. A shift in cell differentiation can be assessed using FACScan analysis of CD surface markers in Flk1+ cells, including CD34, TAL1, CD133, CD45, CD14, Tie-2, VE-cadherin and Sca1. Of selected genes, we generated recombinant viral vectors encoding short interfering hairpin RNAs (siRNA) targeting these genes of interests. Adequate silencing of the targeted gene were verified by RT-QPCR in infected EC. The effect of gene silencing was in addition tested on vascular plexus formation in the 3D EC matrigel model using confocal microscopy and flow cytometry.

Intramuscular injection of recombinant viral vectors encoding selected candidate genes or their matching siRNA, have been utilized to overexpress or silence the gene of interest in murine hind limbs in a standardized mouse model of acute hind limb ischemia. In this ischemia model, the femoral arteries are ligated as described by Couffinhal et al (supra), followed by intramuscular gene transfer to one of the hind limbs, whereas the contra lateral ischemic hind limb served as a control. The effect of sustained expression of angiotrophic or remodeling factors on neovascularization are monitored by quantitative immunohistological analysis (for endothelial, vsmc and inflammatory markers, extracellular matrix synthesis, number and size of capillaries/arterioles in ischemic limb), laser doppler imaging and angiography. To assess the long-term functional and morphological effects of stable expression of angiotrophic factors, animals are sacrificed at 0, 1, 4 and 8 weeks post-gene transfer. In vivo vessel formation are monitored using laser doppler-derived flow measurements, angiography (Rentrop score) and conventional (immuno)histological analysis.

The effect of vasculogenesis regulatory genes specifically expressed in circulating angioblasts, are being assessed in the hind limb ischemia model combined with ex vivo gene transfer in autologous peripheral endothelial progenitors cells. The mononuclear cell fraction is isolated, expanded and transfected with a viral vector encoding selected vasculogenic genes or the targeted siRNA, as well as with a nuclear-tagged beta galactosidase reporter gene prior to intravenous infusion of the autologous cell fraction. In these animals both femoral arteries are ligated. In control animals, the isolated mononuclear cell fraction are likewise harvested, transfected with FIV-ntLacZ alone (in equal tu) and donated. Differentiated EPC (7AAD−/CD45+/CD34+/Flk1+), which are incorporated into newly formed vessels, were identified and quantified using β-galactosidase nuclear staining, double labeled for proliferation markers (Ki67) and differentiation markers (Flk1, CD34, PECAM, Tie2, desmin).

Since the Hmox system was identified as one of the specifically upregulated gene systems in angioblasts during murine embryonic vasculogenesis by our DNAmicroarray and QPCR analysis, we have further studied the role of the heme oxygenase system in vasculogenesis using recombinant viruses encoding Hmox1/2 and heme oxygenase 1 knockout mice, which we have generated. Heme oxygenase (Hmox) is an important regulator of heme biocatalysis and CO production. CO activates guanylate cyclase activity and hence induces cGMP generation, which in turn promotes cGMP dependent kinase activity. The Hmox system is comprised of constitutivelly expressed (Hmox2/3) and inducible isoforms (Hmox1), responsive to multiple stress stimuli. Hmox1-induced CO generation was previously shown to regulate vessel tone and platelet aggregation. In addition, our previous studies suggest that Hmox1 can potently inhibit mitogenesis and arrest vsmc in the G1/G0 phase in vitro and in vivo by upregulation of p21 resulting in a long-term growth arrest. Since inhibition of Hmox1 and the guanylate cyclase pathway led to a reversal of growth inhibition and NOS inhibition failed to normalize growth inhibition, suggests that these effects appear to be distinct from shedding to the NOS/NO^{o} system. We hypothesize that the Hmox/CO and the NOS/NO^{o} system may also have similar yet distinct functions in vasculogenesis and vascular repair response. Both systems activate soluble guanylate, induce cGMP production and activate PKGs. Despite striking similarities, the gaseous NO^{o}/CO second messenger systems also have distinct properties. For instance, Hmox1 is expressed in a tissue specific manner. In the myocardium, NOS cannot be detected and here, Hmox is the main regulator of cGMP production. Under hypoxic conditions, Hmox1, and not NOS, increases cGMP in cultured cardiomyocytes and vsmc. These and preliminary results from our studies suggest that the NOS and Hmox system have complementary functions in the maintenance of cellular homeostasis. In concordance, preliminary in vitro studies show that the mitogenic potential of VEGF in EC cultures is attenuated by the Hmox1 inhibitor SnPPIX, whereas CO release potentiated VEGF release in EC cultures, suggesting that the Hmox/CO system acts downstream of the Flk1 receptor and may be essential to mount an adequate EC mitogenic response elicited by VEGF.

In line with these observations, the heme oxygenase (Hmox) system was indeed identified by our DNAarray analysis as one of the upregulated genes in embryonic and ischemic vasculogenesis. To further elucidate the role of heme oxygenase in embryonic and ischemic (neo)vascularization, we have generated a Hmox1-knockout mouse and recombinant viruses encoding Hmox1/2 and Hmox1/2-silencing siRNA. Hmox1 silencing by siRNA in EC cultures generated an attenuated response to FGF/VEGF stimulation as compared to treatment with a sham virus. We concluded that the heme oxygenase system provides an alternate cytoprotective system permitting endothelial cell quiescence and cytoprotective properties during ischemia and compensatory neovascularization.

The role of the Hmox system have been first studied by analysis of in vitro formation of capillary like structures in primary 3D matrixgel cultures of Hmox1 or 2-overexpressing and Hmox1 or 2-silenced EC following lentiviral gene transfer. Analysis of vascular sprouts were analyzed as described using confocal microscopy and morphometry.

In vivo implications of Hmox1 deletion on vasculogenesis are being tested in the ischemic leg model in Hmox1-nullizygous mice as compared to wild type littermates. Vessel formation are monitored at 0, 1, 2 and 3 weeks following ligation by laser doppler flow imaging, angiography, and quantitative histological analysis as described.

The specific requirement of Hmox in mature vasculogenesis is studied using the acute hind limb ischemia model following bone marrow transplantation from Hmox-deficient donors into wild type littermates. Wild type C57B16 mice are irradiated to deplete the bone marrow, followed by an intravenous infusion of bone marrow cells harvested from Hmox1−/−-littermates. Control animals receive bone marrow from Hmox1+/+ littermates. Animals are allowed to recover for 3 weeks and undergo a bilateral femoral ligation. Neoangiogenesis is tested using laser doppler flow imaging, angiography and confocal microscopy (including Hmox1-immunostaining). This study clarified the relative contribution of Hmox1 expression in circulating angioblasts to the vasculogenesis response.

Expression of the prognostic value of the identified vasculogenesis genes may be further validated using microarray analysis in different subsets of cardiovascular partients and correlated to patient outcome and phenotypes. Using genome wide microarray analysis we are verifying expression of the vasculogenic genes and gene products in different patient groups, and cross correlated to conventional measurements of ischemia and myonecrosis detections and correlate DNA/RNA/protein profile with disease outcome and prognosis. The patient cohorts include, but is not be restricted to patients with stable angina pectoris, unstable angina pectoris, acute coronary syndrome, patients undergoing transient ischemic cerebrovascular events (TIA/CVA), peripheral vascular disease, and patients with refractory angina pectoris. Expression profile of these genes are correlated to (but not limited to): diagnosis, other objectified indicators of ischemia (nuclear perfusion imaging, myonecrosis markers (TropT, CKMB), angiographic analysis), the occurrence of re-events (MACCE), progression of angina pectoris and, efficacy of initiated pharmacotherapy. 1000 cardiovascular patients are being analyzed using a dna microarray analysis including our newly identified vasculogenic candidate genes (involved in ischemia-driven EPC activation, neovascularization, and vascular repair) but also include genes that previously shown to be involved in neovascularization (as a positive control).

These studies seek to delineate the genetic regulation of vasculogenesis by genome-wide analysis of candidate regulatory genes during embryonic and ischemia-related vessel formation. Candidate genes have been assessed using analysis of endogenous expression patterns in mouse development, and have been further explored and selected using cross correlation with expression in zebrafish development, in an adult mouse ischemia models and in human CAD disease. The combination of mouse, zebrafish and human genomics provides an efficient (selection) strategy to obtain functional knock down data in the fish and mice for evolutionary conserved genes, previously identified during mouse and zebrafish development. In vivo tTransgene analysis of vessel formation during zebrafish development and in vitro, in 3D matrigel analysis have further narrowed down the selection of candidate genes to 64 candidate genes, which were further explored in depth in a murine model of ischemic vasculogenesis by viral vector mediated gene transfer analysis. The selected clones comprise among others, the heme oxygenase family. The role of heme oxygenases, as one of the identified regulatory gene families, have been studied in vitro with overexpression or silencing of Hmox family members and in vivo in mouse models of neovascularization using Hmox1-knockout mice and bone marrow transplantations. Additional expression profiling by use of QPCR and microarray analysis in cardiovascular patients further validates the relation between there regulatory genes in vasculogenesis and arterial repair with the progression and prognosis of these patients. These previous studies have provided insight into the molecular mechanisms of vasculogenesis (and arterial repair), and may constitute an attractive novel molecular treatment strategy for cardiovascular ischemic disease.

### Example 3

### Validation of the Genes Cited in Aspects of this Invention in Humans and Experimental Animal Models

The present invention relates to compositions and methods for the diagnostics and prognostics of cardiovascular disease by evaluation of the selected vasculogenenic/angiogenic genes, as previously identified by microarray analysis. In particular, the present invention includes the use of specific gene expression profiles of blood circulatory endothelial progenitor cells to diagnose cardiovascular disease and predict clinical outcome. The feasibility of the methods for this approach are assessed and validated, in both human groups, and in experimental animal models for cardiovascular disease.

### Example 3.1

### Validation of Expression of the Genes in Aspect of this Invention in the Blood Mono-Nuclear Cell Fraction in a Porcine Model for Ischemia

Objective (1): To test if the genes as cited in the present application are indeed expressed in the blood mono-nuclear cell fraction and in endothelial progenitor cells in healthy and post ischemic animals
Objective (2): To test if the blood derived mono-nuclear cell fraction endothelial progenitor cell fraction can be used for gene profiling of the candidate genes
Objective (3): To assess if ischemia induces an alteration in the expression profile of the genes cited in aspects of this invention.
Material and methods: In cardiovascular patients, coronary vessel occlusion accounts for local low oxygen (ischemic) condition in the heart that subsequently results in damage of heart tissue and loss of heart function. Although ischemia accounts for the very onset of heart disease, early ischemic conditions can be quiescent, and therefore escape detection by conventional diagnostic methods. Here we mimic the early onset of ischemia in a well-validated porcine model. In a total of 6 pigs, a light ischemic episode was induced by inflation of a 3 mm diameter balloon catheter in one of the main coronary arteries (LAD) for 6 minutes. Animals were anesthetized by inhalation of isoflurane/oxygen during the procedure, and allowed to recover afterwards. Before and 24 hours after the procedure, 25 ml of venal blood was collected and immediately processed. The mono nuclear fraction was isolated by Ficoll gradient. Briefly, blood samples were pipetted on top of 12.5 ml Ficoll-Paque Plus in 50 ml Falcon tubes, and centrifuged at 2000 rpm to separate the band with mononuclear cells. The mononuclear fraction was collected by pipetting, and the cells were washed twice with ice-cold PBS before 2 ml ACK lysis buffer was added to lyse the remaining erythrocytes. After 2 minutes of incubation at room temperature, the cells were washed twice with ice-cold PBS, and the pellet was treated with RLT lysis buffer (Qiagen) for RNA isolation. Endothelial progenitor cells were selected using flow cytometry cell sorting using 7AAD/CD45/CD34/flk1 selection. RNA isolation was conducted with a commercially available RNA isolation kit (Qiagen). The RNA was reversed transcribed using the Invitrogen reverse transcription kit following the manufacturer's instructions using random hexamers (Invitrogen). We analyzed the gene expression of the genes cited in aspects of this invention using a quantitative (Q)PCR technique. Briefly, primers for the genes cited in aspects of this invention were designed using 3primer software, and QPCR was performed using the Biorad cybergreen detection mix (Biorad) following the manufacturer's protocol. QPCR samples were measured by cybergreen detection by the MyiQ real time PCR detection system (Biorad) and data was subsequently analyzed using the supplied Biorad software. Expression levels were corrected for the expression of the household gene beta-actin.

### Results Objective 1 and 2:

Isolation of adequate amounts of total RNA from the mononuclear fraction derived from blood samples is feasible: on average, >10 microgram of total RNA could be obtained from 25 ml of whole blood. We tested the expression of the following genes: Adora2a, Agtrl1, ets1, Dll4, Lgmn, Rin3, Thsd1, Cngl1, and Elk3. Adora2a, Agtrl1, ets1, Dll4, Lgmn, Rin3, Thsd1, Cngl1, and Elk3 could all be detected in the blood mono-nuclear cell fraction, before and after ischemia induction. QPCR analysis showed that these genes had an average threshold cycle of <30, indicating that these genes were highly expressed in blood mononuclear cells.

Conclusion objective 1 and 2: It is feasible to isolate adequate amounts of RNA from the mononuclear cell fraction from 25 ml of blood. In addition, Adora2a, Agtrl1, ets1, Dll4, Lgmn, Rin3, Thsd1, Cngl1, and Elk3 are detectable in the blood mononuclear cell fraction of healthy and post ischemic groups.
Results objective 3: We observed significant upregulation in the expression of Adora2a (+214±13% versus +31±10%, post and pre ischemia respectively), Agtrl1 (+301±11% versus +14±9%, post and pre ischemia respectively), Dll4 (+152±16% versus +21±5%, post and pre ischemia respectively), Lgmn (+411±31% versus +14±16%, post and pre ischemia respectively), Rin3 (+143±25% versus +6±10%, post and pre ischemia respectively), Thsd1 (+156±5% versus +24±7%, post and pre ischemia respectively), Cngl1 (+199±9% versus +17±14%, post and pre ischemia respectively) and Elk3 (+205±12% versus +1±3%, post and pre ischemia respectively), 24 hours after the ischemic event, whereas ets1 (+15±23% versus +14±9%, post and pre ischemia respectively) expression was not significantly affected. The GAPDH household gene were included as a control. The expression levels of this gene were not significantly affected (+2±12% versus +1±30%, post and pre ischemia respectively).
Conclusion objective 3: Expression levels of Adora2a, Agtrl1, Dll4, Lgmn, Rin3, Thsd1, Cngl1, and Elk3 are highly sensitive biomarkers for the detection of mild ischemia.

### Example 3.2

### Validation of Expression of the Genes Cited in Aspects of this Invention in the Blood Mono-Nuclear Cell Fraction in Healthy Humans and Myocardial Patients

Objective (1): To test if the genes cited in aspects of this invention are indeed expressed in the blood mono-nuclear cell fraction in healthy subjects and acute patients with myocardial infarction.
Objective (2): To test if the blood derived mono-nuclear cell fraction can be used for gene profiling of the candidate genes

### Material and Methods:

A total of 6 patients entering the clinic signs of cardiovascular disease or ongoing ischemia were studied. As a control, a cohort of 6 healthy, randomly selected volunteers in the age between 24 and 60 was included. 50 ml of venal blood was collected and immediately processed. The mononuclear fraction was isolated by Ficoll gradient. Briefly, blood samples were pipetted on top of 12.5 ml Ficoll-Paque Plus in 50 ml Falcon tubes, and centrifuged at 2000 rpm to separate the band with mononuclear cells. The band with the mononuclear fraction was collected by pipetting, and the cells were washed twice with ice-cold PBS before 2 ml ACK lysis buffer was added to lyse the remaining erythrocytes. After 2 minutes of incubation at room temperature, the cells were washed twice with ice-cold PBS, and the pellet was treated with RLT lysis buffer (Qiagen) for RNA isolation. EPCs were selected from the mononuclear cell fraction by flow cytometric cell sorting (FACSdiva, B&D) using immunolabelling for 7AAD/CD45+/CD34+/KDR+. RNA isolation was conducted with a commercially available RNA isolation kit (Qiagen). The RNA was reversed transcribed using the Invitrogen reverse transcription kit following the manufacturer's instructions using random hexamers (Invitrogen). We analyzed the gene expression of the genes cited in aspects of this invention using a quantitative (Q)PCR technique. Briefly, primers for the genes cited in aspects of this invention were designed using 3primer software, and QPCR was performed using the Biorad cybergreen detection mix (Biorad) following the manufacturer's protocol. QPCR samples were measured by cybergreen detection using the MyiQ real time PCR detection system (Biorad) and data was subsequently analyzed using the supplied Biorad QPCR analysis software. Expression levels were corrected for the expression of the household gene beta-actin.

Results objective 1 and 2: Isolation of adequate amounts of total RNA from the mononuclear fraction and endothelial progenitor cell fractions derived from blood samples is feasible: on average, >15 microgram of total RNA from PBMC could be obtained from 50 ml of whole blood. For future microarray analysis, only 1 microgram is needed. 33 genes were tested (including; Sox7, Sox18, Adora2A, Lama4, Lamb1-1, Crip2, Rock2, Rin3, Cgnl1, Fgd5, Elk3, Agtrl1, Apelin, KDR, Ets2, NRP1, NRP2, Notch4, DLL4, Eelk3, Erg1, Stab1, Stab2, Grrp1, Thsd1, HO-1, (Hmox1), HO-2 (Hmox2), Lgmn, Exoc3L, HO-2, PLVAP, Xlkd1, TNFalpha inducible protein 8 (TNFaip8) and showed expression, whereas Rin3, PVLAP, Crip2, Lgmn, NRP2, NRP1, Notch1, Notch4, Sox7, TNFaIP8L1, Elk3, and Sox18 showed high expression levels in mononuclear cell fractions in both healthy human subjects and acute myocardial infarction patients.
Conclusion objective 1 and 2: It is feasible to isolate adequate amounts of RNA for QPCR and future microarray analysis from the mononuclear cell fraction from 50 ml of blood. In addition elevated expression levels of Sox7, Sox18, Adora2A, Lama4, Lamb1-1, Crip2, Rock2, Rin3, Cgnl1, Fgd5, Elk3, Agtrl1, Apelin, KDR, Ets2, NRP1, NRP2, Notch4, DLL4, Eelk3, Erg1, Stab1, Stab2, Grrp1, Thsd1, HO-1, (Hmox1), HO-2 (Hmox2), Lgmn, Exoc3L, HO-2, PLVAP, Xlkd1, TNFalpha inducible protein 8 (TNFaip8) could be detected in acute myocardial infarction patients.

### Example 3.3

### Validation of Two of the Genes Cited in Aspects of this Invention (Agtrl1 and Apelin) in a Well-Validated Murine Model for Ischemia and Myocardial Infarction

### Example of One Gene:

Literature study identified Agtrl1 as a cell membrane receptor for the ligand apelin. Here we assessed:

Objective (1): The expression level of Agtrl1 on the cell membrane of circulatory endothelial progenitor cells in mice, in healthy animals and in response to experimentally induced ischemia and myocardial infarction.
Material and methods: Ischemia in mice was induced by permanent ligation of the femoral artery, which led to subsequent low oxygen conditions in the hind limb muscles. Myocardial infarction in mice was induced by permanent occlusion of one of the main coronary arteries, leading to myocardial infarction. Briefly, c57bl/6 mice were anesthesized by inhalation of isoflurane/oxygen. The femoral artery was dissected and ligated for hind limb ischemia induction, or the thorax was opened and the LAD was dissected and ligated for the induction of a myocardial infarction. The hind limb tissue or thorax was closed and animals were allowed to recover. Animals were sacrificed after 4 days. Blood samples of 1 ml were collected from non-treated control animals, and from animals either subjected to hind limb ischemia or myocardial infarction. The mononuclear fraction was isolated by Ficoll gradient. Briefly, blood samples were pipetted on top of 12.5 ml Rodent Ficoll-Paque Plus in 50 ml Falcon tubes, and centrifuged at 2000 rpm to separate the band with mononuclear cells. The band with the mononuclear fraction was collected by pipetting, and the cells were washed twice with ice-cold PBS before 2 ml ACK lysis buffer was added to lyse the remaining erythrocytes. After 2 minutes of incubation at room temperature, the cells were washed twice with ice-cold PBS. The cells were stained in 200 microliters of FACs buffer using rabbit polyclonal antibodies directed against Agtrl1 (Abcam), and rat monoclonal antibodies directed against c-kit and Flk1 (directly labeled with FITC and APC respectively) for 20 minutes at room temperature. Cells are then washed twice in ice-cold FACS buffer, before incubation with the secondary antibody directed against rabbit IgG, followed by two washes in ice-cold FACs buffer. The pellet is resuspended in 500 microliters of FACs buffer before analysis by flow cytometry on a FACSCanto flow cytometer (BD) followed by analysis using the supplied software. Endothelial progenitor cells were identified using the specific cell membrane markers c-kit+/Sca1+ and Flk1, and Agrtl1 protein expression on the cell membrane of these endothelial progenitor cells was measured by flow cytometry.
Results objective 1: Agrtl1 was highly expressed on a specific subset of endothelial progenitor cells (c-kit/Flk1+) compared with non-relevant white blood cells (4530±312 versus 1211±141, respectively). The number of circulatory Agrtl1-high c-kit/Flk1+ cells was increased in response to myocardial infarction (1.5±0.11% of total cell population versus 4.1±0.31% of total cell population, in the healthy versus the myocardial group respectively), but not in response to ischemia in the hind limb (1.5±0.11% of total cell population versus 1.7±0.44% of total cell population, in the healthy versus the myocardial group respectively).
Conclusion objective 1: Agrtl1 is detectable on the cell membrane of endothelial progenitor cells. In particular, Agtrl1 is highly expressed on cell membrane of the c-kit/Flk1 progenitor cell subpopulation. Myocardial infarction can recruit Agrtl1-high c-kit/Flk1 progenitor cells into the blood circulation. Therefore, these results demonstrate that Agrtl1 is an efficient diagnostic and prognostic marker of myocardial infarction.

The same type of animal studies can be conducted for remaining genes, which all are involved in the process of vasculogenesis in development and are upregulated during this process of vasculogenesis both in development and ischemia driven vasculogenic reponse: including Sox7, Sox18, Adora2A, Lama4, Lamb1-1, Crip2, Rock2, Rin3, Cgnl1, Fgd5, Elk3, Agtrl1, Apelin, KDR, Ets2, NRP1, NRP2, Notch4, DLL4, Eelk3, Erg1, Stab1, Stab2, Grrp1, Thsd1, HO-1, (Hmox1), HO-2 (Hmox2), Lgmn, Exoc3L, HO-2, PLVAP, Xlkd1, TNFalpha inducible protein 8 (TNFaip8) and TNFaip8l1. The candidate genes are involved in various facets of vasculogenic approach including angioblast migration, vessel permeabilization, EPC chemotaxis and EPC survival and differentiation, including ateriovenous differentiation. They all appear to be involved in the vasculogenesis response in development as well as in the adult. Currently, FGD5, TNFaIP8L1, rin3, Thsd1, stab1, stab2, sox7, sox18, GGRP, Agtrl/apelin, Hmox1/2 and PLVAP among other are extensively studied in various animal models and human patients.

1-30. (canceled)

31. A method of diagnosis or prognosis of cardiovascular disease in a subject, said method comprising the steps of detecting the presence of activated endothelial progenitor cells (EPCs) in a sample of a circulation fluid of said subject.

32. Method according to claim 31, wherein said cardiovascular disease is associated with arterial damage or myocardial damage.

33. Method according to claim 31, wherein said cardiovascular disease is associated with ischemia.

34. Method according to claim 31-32 or 33, wherein said step of detecting activated EPCs comprises the detection in said sample of an increase in the gene expression level in EPCs of at least one gene and even more preferably at least 2, 3, 4, 5, 10, 15, 20, 25, 30, 35, or all genes selected from the group consisting of ADORA1, ADORA2A, ADORA2B, ADORA3, AGTRL1 (APLNR), AMPH, APLN, CCBE1, CDC42, CGNL1, CREBBP, CRIP1, CRIP2, CRIP3, CYB5B, DLL4, DUSP5, EEA1, egr-1, ELK1, ELK3, ELK4 (SAP1), EP300, ERG1 (KCNH2), ETS1, ETS2, EXOC3L, FGD1, FGD2, FGD3, FGD4, FGD5, FLT1, FST, GATA6, GRRP1, HO-1 (HMOX1), HO-2 (HMOX2), IFNG, IL1A, IL1B, LAMA4, Lamb1-1, LGMN, MMP3, Nos2, PAI1, PHD1, PLVAP, RAB5a, RIN3, ROCK2, SOX18, SOX7, SRF, STAB1, STAB2, STUB1, TFEC, THBS1, THBS2, THBS3, THBS4, THBS5, THSD1, TNFAIP8, and XLKD1 (LYVE1), still preferably at least one gene and yet still more preferably at least 2, 3, 4, 5, 10, 15, 20, 25, 30 or all genes selected from the group consisting of ADORA2A, AGTRL1 (APLNR), APLN, CCBE1, CGNL1, CRIP2, CYB5B, DLL4, DUSP5, ELK3, ERG1 (KCNH2), ETS1, ETS2, EXOC3L, FGD5, GRRP1, HO-1 (HMOX1), HO-2 (HMOX2), LAMA4, Lamb1-1, LGMN, PLVAP, RIN3, ROCK2, SOX7, SOX18, STAB1, STAB2, STUB1, TFEC, THSD1, TNFAIP8, and XLKD1 (LYVE1).

35. Method according to claim 34, wherein said increase in the gene expression level in EPCs is detected by detection of a protein.

36. A biomarker for diagnosis or prognosis of cardiovascular disease in a patient, said biomarker comprising the expression product of a gene of an endothelial progenitor cell (EPC) the expression of which is regulated during vasculogenesis.

37. The biomarker of claim 36, wherein said biomarker comprises the expression product of a gene of an endothelial progenitor cell (EPC) the expression of which is upregulated during vasculogenesis compared to angiogenesis.

38. The biomarker of claim 36 or 37, wherein the biomarker is present in endothelial progenitor cells (EPCs), polymorphonuclear leukocytes (PMNs) or whole blood.

39. The biomarker of claim 33, wherein said EPCs or PMNs are present in the peripheral blood of patients.

40. The biomarker of claim 33, wherein said EPCs are Flk1-positive cells.

41. The biomarker of claim 36 or 37, wherein said biomarker is an expression product of at least one gene and even more preferably at least 2, 3, 4, 5, 10, 15, 20, 25, 30, 35, or all genes selected from the group consisting of ADORA1, ADORA2A, ADORA2B, ADORA3, AGTRL1 (APLNR), AMPH, APLN, CCBE1, CDC42, CGNL1, CREBBP, CRIP1, CRIP2, CRIP3, CYB5B, DLL4, DUSP5, EEA1, egr-1, ELK1, ELK3, ELK4 (SAP1), EP300, ERG1 (KCNH2), ETS1, ETS2, EXOC3L, FGD1, FGD2, FGD3, FGD4, FGD5, FLT1, FST, GATA6, GRRP1, HO-1 (HMOX1), HO-2 (HMOX2), IFNG, IL1A, IL1B, LAMA4, Lamb1-1, LGMN, MMP3, Nos2, PAI1, PHD1, PLVAP, RAB5a, RIN3, ROCK2, SOX18, SOX7, SRF, STAB1, STAB2, STUB1, TFEC, THBS1, THBS2, THBS3, THBS4, THBS5, THSD1, TNFAIP8, and XLKD1 (LYVE1), still preferably at least one gene and yet still more preferably at least 2, 3, 4, 5, 10, 15, 20, 25, 30 or all genes selected from the group consisting of ADORA2A, AGTRL1 (APLNR), APLN, CCBE1, CGNL1, CRIP2, CYB5B, DLL4, DUSP5, ELK3, ERG1 (KCNH2), ETS1, ETS2, EXOC3L, FGD5, GRRP1, HO-1 (HMOX1), (HMOX2), LAMA4, Lamb1-1, LGMN, PLVAP, RIN3, ROCK2, SOX7, SOX18, STAB1, STAB2, STUB1, TFEC, THSD1, TNFAIP8, and XLKD1 (LYVE1).

42. The biomarker of claim 41, wherein said expression product is a protein or RNA molecule.

43. The biomarker of claim 41, wherein said biomarker is a protein profile or RNA profile.

44. Use of a biomarker as defined in claim 36 for the diagnosis or prognosis of ischemia in a subject.

45. Use of a biomarker according to claim 36 as a surrogate end-point marker for determining the efficacy of therapeutic methods.

46. A method for the diagnosis or prognosis of ischemia in a subject, comprising detecting in the blood of said subject a biomarker according to claim 36.

47. The method of claim 46, wherein said method is performed on a sample of blood of said subject.

48. The method of claim 46 or 47, wherein said detection is performed by using microarrays.

49. The method of claim 46 or 47, wherein said detection is performed by using tandem mass spectrometry (MS-MS), by MALDI-FT mass spectrometry, MALDI-FT-ICR mass spectrometry, MALDI Triple-quad mass spectrometry or immunoassay.

50. Kit of parts comprising at least one biomarker as defined in claim 36 or a specific binding partner that binds specifically to said biomarker, said kit of parts optionally further comprising one or more of the following:
at least one reference or control sample;
information on the reference value for the biomarker;
at least one test compound capable of binding to said specific binding partner;
at least one detectable marker for detecting binding between said biomarker and said specific binding partner.

51. A microarray for performing a method according to claim 46, comprising specific binding partners that bind specifically to at least two biomarkers as defined in claim 36 bound to a solid support.

52. A diagnostic reagent that binds specifically to a biomarker as defined in claim 36.

53. The diagnostic reagent of claim 52, wherein said diagnostic reagent is an antibody or a nucleic acid molecule specifically hybridizing under stringent conditions to said biomarker.

54. A diagnostic composition comprising a diagnostic reagent according to claim 52 or 53.

55. Use of a diagnostic composition according to claim 54, for diagnosing ischemia in a subject.

56. A method of treating a subject (having an increased risk of) suffering from ischemia, said method comprising using a biomarker as defined in any one of claim 36 or 37 as a therapeutic target or as a therapeutic agent.

57. The method of claim 56, wherein said use of said biomarker as a therapeutic target or as a therapeutic agent comprises affecting in said subject the expression of at least one gene and even more preferably at least 2, 3, 4, 5, 10, 15, 20, 25, 30, 35, or all genes selected from the group consisting of ADORA1, ADORA2A, ADORA2B, ADORA3, AGTRL1 (APLNR), AMPH, APLN, CCBE1, CDC42, CGNL1, CREBBP, CRIP1, CRIP2, CRIP3, CYB5B, DLL4, DUSP5, EEA1, egr-1, ELK1, ELK3, ELK4 (SAP1), EP300, ERG1 (KCNH2), ETS1, ETS2, EXOC3L, FGD1, FGD2, FGD3, FGD4, FGD5, FLT1, FST, GATA6, GRRP1, HO-1 (HMOX1), HO-2 (HMOX2), IFNG, IL1A, IL1B, LAMA4, Lamb1-1, LGMN, MMP3, Nos2, PAI1, PHD1, PLVAP, RAB5a, RIN3, ROCK2, SOX18, SOX7, SRF, STAB1, STAB2, STUB1, TFEC, THSD1, THBS2, THBS3, THBS4, THBS5, THSD1, TNFAIP8, and XLKD1 (LYVE1), still preferably at least one gene and yet still more preferably at least 2, 3, 4, 5, 10, 15, 20, 25, 30 or all genes selected from the group consisting of ADORA2A, AGTRL1 (APLNR), APLN, CCBE1, CGNL1, CRIP2, CYB5B, DLL4, DUSP5, ELK3, ERG1 (KCNH2), ETS1, ETS2, EXOC3L, FGD5, GRRP1, HO-1 (HMOX1), HO-2 (HMOX2), LAMA4, Lamb1-1, LGMN, PLVAP, RIN3, ROCK2, SOX7, SOX18, STAB1, STAB2, STUB1, TFEC, THSD1, TNFAIP8, and XLKD1 (LYVE1).

58. The method of claim 56, wherein said use of said biomarker as a therapeutic target comprises decreasing the amount of at least one protein encoded by said at least one gene that is over-expressed in subjects (having an increased risk of) suffering from ischemia, or increasing the amount of at least one protein encoded by said at least one gene that is under-expressed in subjects (having an increased risk of) suffering from ischemia.

59. Pharmaceutical composition for the treatment of an increased risk of suffering a cardiovascular event, comprising at least one inhibitor compound selected from:
an antibody or derivative thereof directed against the biomarker of claim 36, preferably a biomarker expressed on the cell membrane, and said derivative preferably being selected from the group consisting of scFv fragments, Fab fragments, chimeric antibodies, bifunctional antibodies, intrabodies, and other antibody-derived molecules;
a biomarker of any one of claim 36,
a small molecule interfering with the biological activity of said biomarker.
an antisense molecule, in particular an antisense RNA or antisense oligodeoxynucleotide,
an RNAi molecule,
a ribozyme, and
a chemical compound interfering with the function of the designated markers/regulatory genes
and a suitable excipient, carrier or diluent.

60. Method of treating a subject, comprising administering to said subject the pharmaceutical composition of claim 59 in an amount effective to decrease (the risk of) ischemia.
